(19) United States
(12) Patent Application Publication
(10) Pub. No.: US 2026/0273263 A1
(43) Pub. Date: Sep. 17, 2026

(54) TRANSCATHETER IMPLANTABLE BLOOD PUMP AND METHODS THEREFOR

(71) Applicant: Puzzle Medical Devices Inc., Montreal (CA)

(72) Inventors: François TRUDEAU, Montreal (CA); Yves-Antoine CRÊTE, Montreal (CA); Gabriel GEORGES, Montreal (CA); Philippe CORMIER, Montreal (CA); Marc BLONDIN, Montreal (CA); Maxime ROCHON, Montreal (CA); Daniel OYAMA, Montreal (CA); Jack POULTON, Montreal (CA)

(21) Appl. No.: 19/677,960

(22) Filed: May 14, 2026

Related U.S. Application Data
(63) Continuation of application No. PCT/CA2026/050343, filed on Mar. 4, 2026.
(60) Provisional application No. 63/766,821, filed on Mar. 4, 2025.
(60) Provisional application No. 63/775,918, filed on Mar. 21, 2025.
(60) Provisional application No. 63/815,182, filed on May 30, 2025.
(60) Provisional application No. 63/841,077, filed on Jul. 9, 2025.
(60) Provisional application No. 63/905,234, filed on Oct. 24, 2025.
(60) Provisional application No. 63/922,947, filed on Nov. 21, 2025.

(51) Int. Cl. A61M 60/242; A61M 60/139; A61M 60/411; A61M 60/419; A61M 60/806; A61M 60/81; A61M 60/824; A61M 60/865

(52) U.S. Cl. CPC A61M 60/242; A61M 60/139; A61M 60/411; A61M 60/419; A61M 60/806; A61M 60/81; A61M 60/824; A61M 60/865

(57) ABSTRACT

A transcatheter blood pump implantable within a subject's lumen defined by a subject's lumen wall. The transcatheter blood pump includes an impeller having an axis of rotation, a shroud having a first shroud end portion, a second shroud end portion, and a shroud internal passage extending between the first shroud end portion and the second shroud end portion, the impeller disposed within the shroud internal passage, an electric motor configured to rotate the impeller, the electric motor having a first motor end portion and a second motor end portion, the first motor end portion coupled to the second shroud end portion, and an electric driveline configured to transmit operating power to the electric motor to rotate the impeller. The electric driveline extends along at least one of the shroud and the electric motor and is coupled thereto.

### FIELD

This disclosure relates generally to a transcatheter implantable blood pump and methods therefor.

### BACKGROUND

Transcatheter implantable blood pumps have been developed to provide mechanical circulatory support using minimally invasive delivery techniques. Such devices may be delivered through the vasculature and positioned within a subject's lumen, such as within a blood vessel or the aorta, where a rotating pump element operates to propel blood in a desired direction.

Despite advances in these technologies, challenges remain in the design and operation of transcatheter implantable blood pumps. In particular, such devices operate within the confined and dynamic environment of the vasculature during implantation and use. Limitations in pump design, device positioning, interaction with blood flow, and interaction with surrounding anatomical structures may negatively affect overall device performance and reliability.

For example, inadequate hydrodynamic performance may limit the ability of a pump to generate sufficient fluid outflow. In addition, inadequate stabilization of the device within the subject's lumen may lead to displacement, migration, or undesired orientation changes during operation or subject movement. Furthermore, blood-contacting components of the pump, including rotating elements and bearing structures, may contribute to thrombogenesis under certain conditions.

Accordingly, there remains a need for improved transcatheter implantable blood pumps.

### SUMMARY

An aspect of this disclosure relates to a transcatheter blood pump implantable within a subject's lumen defined by a subject's lumen wall. The transcatheter blood pump including:
an impeller defining an impeller rotation axis;
a shroud having a first shroud end portion, a second shroud end portion, and a shroud internal passage extending between the first shroud end portion and the second shroud end portion, the impeller being disposed within the shroud internal passage;
an electrical drive configured to rotate the impeller, the electrical drive having a first drive end portion and a second drive end portion, the first drive end portion being coupled to the second shroud end portion; and
an electrical driveline configured to transmit operating power to the electrical drive to rotate the impeller,
wherein the electrical driveline extends along at least one of the shroud and the electrical drive and is coupled thereto.

The shroud may include a shroud inlet disposed at the first shroud end portion, and a shroud outlet disposed at the second shroud end portion; the electrical driveline extending along a portion of the shroud defining the shroud outlet and being connected thereto.

The shroud may include a plurality of outlet arm members defining a plurality of shroud sub-outlets therebetween, the shroud sub-outlets being in fluid communication with the shroud outlet, the electrical driveline extending along at least one outlet arm member of the plurality of outlet arm members and being connected thereto.

The impeller may include a downstream taper surface disposed at an axially downstream end of the impeller and oriented obliquely relative to the impeller rotation axis, the downstream taper surface being configured to redirect at least a portion of blood flow radially outward relative to the impeller rotation axis through the shroud outlet.

The electrical driveline may extend along a portion of the shroud defining the shroud inlet and is connected thereto.

The shroud may include a plurality of outlet arm members defining a plurality of shroud sub-outlets therebetween, the shroud sub-outlets being in fluid communication with the shroud outlet, the electrical driveline extending along at least one outlet arm member of the plurality of outlet arm members and being connected thereto.

The shroud inlet may define an inlet opening substantially centered about the impeller rotation axis and configured to direct blood axially relative to the impeller rotation axis into the shroud internal passage.

The electrical driveline may extend along the electrical drive and is connected thereto.

The electrical driveline may be configured to operatively connect to at least one of the first drive end portion and the second drive end portion to transmit operating power to the electrical drive to rotate the impeller.

The transcatheter blood pump implantable may include a first contact element extending from the second drive end portion, the first contact element including at least two atraumatic contact portions configured to atraumatically contact the subject's lumen wall, the first contact element being configured to transition between a delivery configuration for advancing the transcatheter implantable blood pump within the subject's lumen, and a contact configuration in which the at least two atraumatic contact portions are configured to atraumatically contact the subject's lumen wall within the subject's lumen.

The first contact element may not define any portion of the shroud internal passage.

The first contact element may include a terminal portion and a deflection portion extending between the second drive end portion and the terminal portion; the deflection portion being configured to transition the terminal portion between a first terminal portion configuration in which the terminal portion is disposed at a location axially distal relative to the electrical drive along a longitudinal drive axis defined by the electrical drive, and a second terminal portion configuration in which the terminal portion is disposed at a location radially outward relative to the longitudinal drive axis.

The deflection portion may include a segment extending substantially coaxially from the second drive end portion along the longitudinal drive axis.

The terminal portion may include a capturable element configured to be captured by a retrieval instrument within the subject's lumen.

The capturable element may be disposed at a distal-most end of the terminal portion.

The first contact element may include a transition segment configured to maintain the capturable element spaced apart from the subject's lumen wall for capturing the capturable element within the subject's lumen.

The transcatheter blood pump implantable may include a second contact element extending from the first shroud end portion, the second contact element including at least one atraumatic contact portion configured to atraumatically contact the subject's lumen wall.

The second contact element may include a contact portion and a positioning portion extending between the first shroud end portion and the contact portion; the positioning portion being configured to position the contact portion at a location axially distal relative to the shroud along a longitudinal shroud axis defined by the shroud.

The positioning portion may include a segment extending substantially coaxially from the first shroud end portion along the longitudinal shroud axis.

At least one of the positioning portion and the contact portion may include a portion of the electrical driveline.

The electrical driveline portion may be a first electrical driveline portion, the electrical driveline may include a second electrical driveline portion operatively connectable to the first electrical driveline portion, an operative connection between the first electrical driveline portion and the second electrical driveline portion being disposed at the contact portion.

The first contact element and the second contact element may be configured such that a common longitudinal axis defined by the shroud and the electrical drive is oriented at a non-zero angle relative to a longitudinal axis defined by the subject's lumen.

The impeller may include an impeller hub inlet, an impeller hub outlet, and an impeller internal passage in fluid communication with the impeller hub inlet and the impeller hub outlet, the transcatheter blood pump may include:
a hydrodynamic bearing including a first bearing member and a second bearing member;
the first bearing member being coupled to the impeller and defining a first bearing surface, the impeller hub outlet opening onto the first bearing surface, the first bearing member further including a plurality of vanes defining a plurality of vane passages in fluid communication with the impeller internal passage; and
the second bearing member being coupled to the electrical drive and defining a second bearing surface,
wherein rotation of the impeller generates blood flow through the impeller internal passage and through the plurality of vane passages to generate hydrodynamic support between the first bearing member and the second bearing member.

At least one vane of the plurality of vanes may extend outward from the impeller hub outlet toward an outer periphery of the first bearing surface.

At least two vanes of the plurality of vanes may define a plurality of spaced segments of the first bearing surface therebetween.

The second bearing surface may be continuous and free of vane-defined segments.

The first bearing member may define a first bearing surface including a first curved portion and a first flat portion, and the second bearing member may define a second bearing surface including a second curved portion and a second flat portion.

The first flat portion may extend radially outward from the first curved portion, and the second flat portion may extend radially outward from the second curved portion.

The transcatheter blood pump may include a first magnetic element coupled to the impeller, and a second magnetic element coupled to the electrical drive; the first magnetic element being configured to magnetically couple with the second magnetic element to transmit torque between the impeller and the electrical drive, the first magnetic element being disposed at least partially within at least one vane of the plurality of vanes.

The first magnetic element may be at least partially disposed beneath the first curved portion.

Another aspect of this disclosure relates to a transcatheter implantable blood pump, including:
a pump unit;
a drive unit configured to drive the pump unit, the drive unit defining a longitudinal pump unit axis; and
a driveline configured to transmit operating power to the drive unit to drive the pump unit,
wherein the driveline is associated with the pump unit.

Another aspect of this disclosure relates to a transcatheter implantable medical device, including:
a device body having a first body end portion and a second body end portion, and defining a device body longitudinal axis; and
a first contact element extending from the second body end portion, the first contact element including at least two atraumatic contact portions configured to atraumatically contact a subject's lumen wall of a subject's lumen of a subject's body.

Another aspect of this disclosure relates to a transcatheter implantable blood pump, including:
an impeller defining an impeller internal passage extending at least partially between a first impeller end portion and a second impeller end portion thereof;
a drive configured to rotate the impeller; and
a first hydrodynamic bearing including an impeller vaned bearing member and a drive bearing member; the impeller vaned bearing member including a base surface and a plurality of vanes extending therealong; the plurality of vanes defining a plurality of vane passages in fluid communication with a port of the impeller internal passage opening onto the base surface,
wherein rotation of the impeller generates blood flow through the impeller internal passage and through the plurality of vane passages to generate hydrodynamic support between the impeller vaned bearing member and the drive bearing member.

Another aspect of this disclosure relates to a method of implanting a medical device within a subject's body. The method including:
advancing the medical device through a first access site of the subject's body and into a subject's lumen of the subject's body,
wherein a first device portion of the medical device is externalized at least partially through a second access site of the subject's body, and a second device portion of the medical device is positioned at an implantation site within the subject's lumen.

Another aspect of this disclosure relates to a method of explanting a medical device from a subject's body, wherein the medical device comprises a first device portion implanted in a subject's lumen of the subject's body, and a second device portion extending from the first device portion within the subject's lumen and externalized through a second access site of the subject's body. The method including:
establishing a first access site in communication with the subject's lumen; and
retrieving the first device portion from the subject's lumen through the first access site,
wherein the second device portion is internalized through the second access site and retrieved through the first access site.

Features and advantages of the disclosed subject-matter will become apparent in view of the following detailed description of selected embodiments, as illustrated in the accompanying drawings.

### BRIEF DESCRIPTION OF THE DRAWINGS

In order that this disclosure be readily understood, at least some selected embodiments thereof are illustrated by way of example(s) in the drawings. Accordingly, the drawings are illustrative in nature only and are not intended to be construed and interpreted as limiting the extent of the subject matter protected by the claims. Similarly, any text that might appear in any drawings is illustrative in nature only and is not intended to necessitate that any component needs to be included in the embodiment.

It is noted that like reference characters identify similar or equivalent feature(s) throughout the drawings. If present in the claims, reference character(s) is/are provided only to make claims easier to comprehend and are not intended to be construed and interpreted as limiting the extent of the subject matter protected by the claims. Also, the features illustrated throughout the drawings are not necessarily drawn to scale.

FIG. 1 is a schematic representation of a transcatheter implantable blood pump, according to an aspect of this disclosure.

FIG. 2 is a side view of a transcatheter implantable blood pump, such as one of FIG. 1, according to at least one embodiment.

FIG. 3 is a side view of the transcatheter blood pump implantable of FIG. 2, according to at least one embodiment.

FIG. 4 is a perspective view of a shroud of the transcatheter blood pump implantable of FIG. 2, according to at least one embodiment.

FIG. 5 is a side view of the transcatheter implantable blood pump of FIG. 2 illustrating blood flow, according to at least one embodiment.

FIG. 6 is a side view of the transcatheter blood pump implantable of FIG. 2, according to at least one embodiment.

FIG. 7 is a perspective view of the transcatheter blood pump implantable of FIG. 2, according to at least one embodiment.

FIG. 8 is a schematic representation view of two designs of transcatheter implantable blood pumps, including the transcatheter blood pump implantable of FIG. 2, according to an aspect of this disclosure.

FIG. 9 is a side view of the transcatheter blood pump implantable of FIG. 2, according to at least one embodiment.

FIG. 10 is a side view of the transcatheter blood pump implantable of FIG. 2 contained in a medical sheath, according to at least one embodiment.

FIG. 11 is a side view of the transcatheter blood pump implantable of FIG. 2 within a subject's lumen, according to at least one embodiment.

FIG. 12 is a distal view of the transcatheter blood pump implantable of FIG. 2 within a subject's lumen, according to at least one embodiment.

FIG. 13 is a side view of the transcatheter blood pump implantable of FIG. 2 being captured, according to at least one embodiment.

FIG. 14 is a side view of a first contact element of the transcatheter blood pump implantable of FIG. 2, according to at least one embodiment.

FIG. 15 is a side view of a first contact element of the transcatheter blood pump implantable of FIG. 2, according to at least one embodiment.

FIG. 16 is a side view of a second contact element of the transcatheter blood pump implantable of FIG. 2, according to at least one embodiment.

FIG. 17 is a side view of a first hydrodynamic bearing of the transcatheter blood pump implantable of FIG. 2, according to at least one embodiment.

FIG. 18 is a side perspective view of an impeller of the transcatheter blood pump implantable of FIG. 2, according to at least one embodiment.

FIG. 19 is a side perspective view of the impeller of FIG. 18, according to at least one embodiment.

FIG. 20 is a side perspective view of an impeller of the transcatheter blood pump implantable of FIG. 2, according to at least one embodiment.

FIG. 21 is a perspective view of a concave bearing surface provided to an impeller of the transcatheter blood pump implantable of FIG. 2, according to at least one embodiment.

FIG. 22 is a perspective view of a convex bearing surface provided to a drive unit of the transcatheter blood pump implantable of FIG. 2, according to at least one embodiment.

FIG. 23 is a perspective view of a concave bearing surface provided to an impeller of the transcatheter blood pump implantable of FIG. 2, according to at least one embodiment.

FIG. 24 is a bottom view of a concave bearing surface provided to an impeller of the transcatheter blood pump implantable of FIG. 2, according to at least one embodiment.

FIG. 25 is a bottom view of a concave bearing surface provided to an impeller of the transcatheter blood pump implantable of FIG. 2, according to at least one embodiment.

FIG. 26 is a transverse view of an impeller of the transcatheter blood pump implantable of FIG. 2, according to at least one embodiment.

FIG. 27 is a transverse view of an impeller, a shroud, and a magnetic coupling system of the transcatheter blood pump implantable of FIG. 2, according to at least one embodiment.

FIG. 28 is a side perspective view of a concave bearing surface provided to an impeller of the transcatheter blood pump implantable of FIG. 2 integrating components of the magnetic coupling system of FIG. 27, according to at least one embodiment.

FIG. 29 is a side perspective view of a shroud of the transcatheter blood pump implantable of FIG. 2 and components of the magnetic coupling system of FIG. 27, according to at least one embodiment.

FIGS. 30-37 are a schematic representation of a method of implanting a medical device within a subject's body, according to an aspect of this disclosure.

FIGS. 38-42 is a schematic representation of a method of explanting a medical device from a subject's body, according to an aspect of this disclosure.

### DETAILED DESCRIPTION

The subject matter of this disclosure is described and explained in the following detailed description with reference to the non-limiting aspect(s), embodiment(s), example(s), element(s), and step(s) (also collectively referred to herein as “features”), as the case may be, presented herein and illustrated in the accompanying non-limiting drawings. The skilled addressee will readily recognize that one or more features can be combined, in whole or in part, as the case may be, even if they are all not explicitly presented and stated herein. Recognizing that these features may vary, the skilled addressee will also readily recognize that any other variants thereof and any combination of these other variants, as the case may be, are contemplated without departing from the scope of this disclosure, even if they are all not explicitly presented and stated herein.

Therefore, these features thereof are intended merely to facilitate an understanding of ways in which the claimed subject matter may be reduced to practice by the skilled addressee. Accordingly, these features shall not to be construed as limiting the scope of the claimed subject matter, which is defined solely by the accompanying claims and applicable law.

The terminology used herein is only for the purpose of describing and explaining the claimed subject matter and is not intended to limit the scope hereof. Unless defined otherwise, all technical, engineering, scientific, and other relevant terminology used herein have the same meanings as commonly understood by the skilled addressee.

As applicable, the headings, section titles, and subsection labels used throughout this disclosure are provided solely for organizational and convenience purposes to facilitate understanding. Such headings and structural divisions are not intended to, and shall not be construed to, define, limit, characterize, or otherwise affect the scope of the subject matter disclosed or claimed.

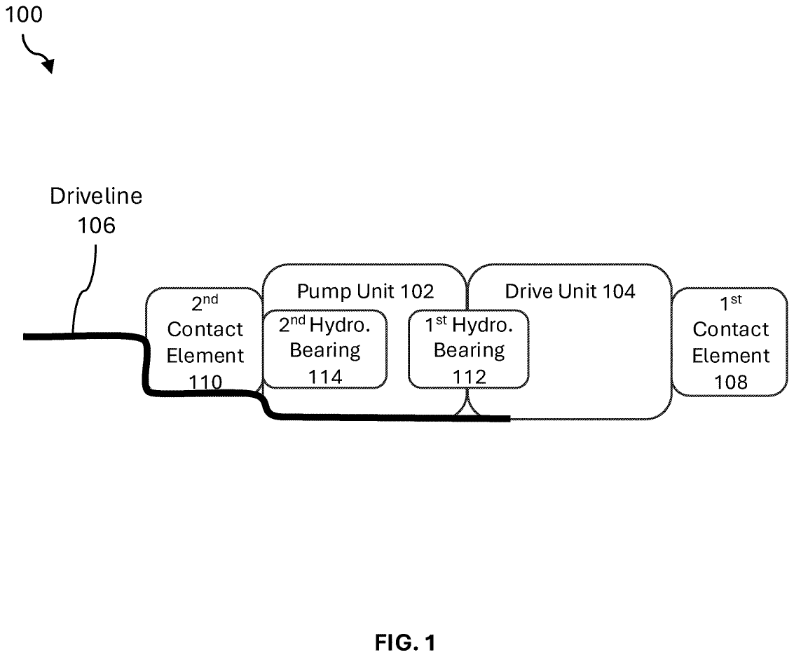

According to an aspect, as schematically represented in FIG. 1, this disclosure relates to a transcatheter implantable blood pump 100 (also referred to herein as a “transcatheter blood pump” or a “transcatheter implantable medical device”) including, for example, a pump unit 102 and a drive unit 104 operatively coupled to the pump unit 102 (the pump unit 102 and the drive unit 104 are herein collectively referred to as a “device body 118”). The pump unit 102 may include a shroud (not shown in FIG. 1) coupled to the drive unit, and an impeller (not shown in FIG. 1) at least partially enclosed within the shroud and rotatable by the drive unit. The drive unit 104 may include an electric motor 204 or an electric actuator.

The transcatheter implantable blood pump 100 optionally includes: a driveline 106 (also referred to herein as a “driveline component”), a first contact element 108, a second contact element 110, a first hydrodynamic bearing 112, and a second hydrodynamic bearing 114. The driveline 106 may be associated at least partially with the pump unit 102 and/or the drive unit 104, for example by extending therealong and being coupled thereto. The driveline 106 may be mechanically coupled to the pump unit 102 and/or the drive unit 104 and may be operatively connected to the drive unit to transmit operating power to the drive unit for operating the pump unit. The driveline 106 may be an electrical driveline, such as an electric cable or a flexible printed circuit board. The driveline 106 may be a mechanical driveline, such as a driveshaft, rotatably coupled to a pump unit 102, in which case the drive unit 104 is not required.

The first hydrodynamic bearing 112 rotatably support an impeller within the shroud. The first contact element 108 extends distally from the drive unit 104, and the second contact element 110 extends proximally from the pump unit 102. The first and second contact elements 108, 110 collectively immobilize and/or stabilize the transcatheter implantable blood pump 100 within a subject's lumen, for example within the lumen of the human aorta.

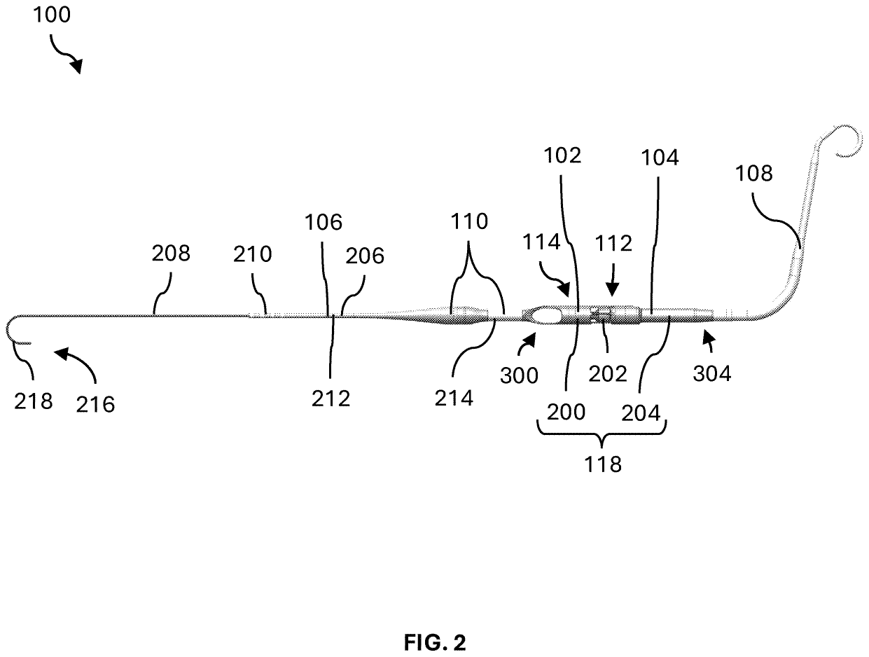

As used herein, the terms “proximal” and “distal” are defined relative to an operator during use of the transcatheter implantable blood pump 100, with “proximal” referring to a direction toward the operator and “distal” referring to a direction away from the operator. Accordingly, for the sake of simplicity, the operator is considered to be on the left in FIG. 2.

FIG. 2 illustrates the transcatheter implantable blood pump 100 including: (i) the pump unit 102, which is optionally provided as a shroud 200 and an impeller 202 disposed within the shroud 200; (ii) the drive unit 104, which is optionally provided as an electric motor 204; the driveline 106, which is optionally provided as an electrical driveline 206; a guidewire 208; the first contact element 108; the second contact element 110; the first hydrodynamic bearing 112 (not clearly visible in FIG. 2), and the second hydrodynamic bearing 114 (not clearly visible in FIG. 2), according to at least one embodiment. The shroud 200, the impeller 202, and the electric motor 204 are herein collectively referred to as the “device body 118”.

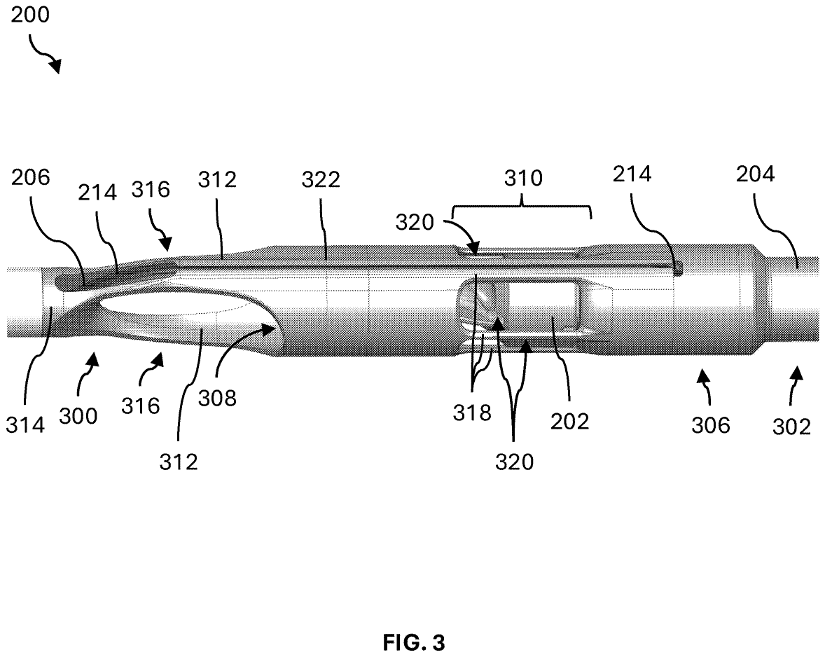

As illustrated in FIGS. 2-3, a second shroud end portion 306 of the shroud 200 is connected to a first motor end portion 302 of the electric motor 204. The impeller 202 is at least partially enclosed within the shroud 200 and is rotatable by the electric motor 204. The electrical driveline 206 is electrically connected to the electric motor 204 to transmit electrical power thereto for rotating the impeller 202. The first contact element 108 extends distally away from a second motor end portion 304 of the electric motor 204. The second contact element 110 extends proximally away from a first shroud end portion 300 of the shroud 200. The electrical driveline 206 extends along at least a portion of the shroud 200 and may also extend along at least a portion of the electric motor 204. The electrical driveline 206 extends away from the shroud 200 and is optionally provided to the second contact element 110. The electrical driveline 206 may further include an electrical connector 210. The guidewire 208 extends from the electrical driveline 206, for example from the electrical connector 210.

With reference to FIG. 2, it will be understood that the first and second shroud end portions 300, 3006 of the shroud 200 correspond to the first and second pump end portions of the pump unit 102, respectively. The first and second motor end portions 302, 304 of the electric motor 204 correspond to the first and second drive end portions of the drive unit 104.

Referring to FIG. 3, the shroud 200 defines a shroud inlet 308 (also referred to herein as a “pump unit inlet” or “port”), a shroud outlet 310 (also referred to herein as a “pump unit outlet” or “port”) and a shroud internal passage (not entirely visible in FIG. 3 and also referred to herein as a “pump unit inner passage”) extending therebetween, according to at least one embodiment. The shroud inlet 308 is disposed at the first shroud end portion 300, and the shroud outlet 310 is disposed at the second shroud end portion 306. The impeller 202 is at least partially disposed within the shroud internal passage.

The shroud 200 optionally includes two inlet arm members 312 disposed at the first shroud end portion 300 and extending proximally therefrom toward one another to converge at a junction body 314, such that the shroud inlet 308 defines two shroud sub-inlets 316 therebetween (also referred to herein as “pump unit sub-inlets”). Each inlet arm member 312 may be profiled, including at least an inner surface thereof facing the shroud internal passage, to improve inlet hydrodynamic performance and to function as a flow straightener promoting laminar inflow. Each inlet arm member 312 may be profiled, including at least an outer surface thereof, to be atraumatic upon introduction of the transcatheter implantable blood pump 100 through a subject's access site and during advancement within a subject's lumen.

Advantageously, the two inlet arm members 312 defining the two shroud sub-inlets 316 prevent or mitigate suction-induced lumen wall apposition at the shroud inlet 308 when the transcatheter implantable blood pump 100 is implanted within a subject's lumen. In particular, continued blood intake through the shroud inlet 308 may be maintained via a first one of the two shroud sub-inlets 316 when a second one of the two shroud sub-inlets 316 is partially or completely occluded, for example by a subject's lumen wall. In such circumstances, the opening of the first shroud sub-inlet may face the subject's lumen wall, while the opening of the second shroud sub-inlet may be oriented away from the lumen wall and toward a subject's central lumen.

The shroud 200 optionally includes five outlet arm members 318 (only some are visible and referred to in FIG. 3) disposed at the second shroud end portion 306 and defining five shroud sub-outlets 320 therebetween (also referred to herein as a “pump unit sub-outlets”; only some are visible and referred to in FIG. 3). Each outlet arm member 318 is optionally profiled, including at least a surface thereof facing the shroud internal passage and impeller 202, to improve inlet hydrodynamic performance, and may be configured to function as a flow straightener to promote laminar inflow. For example, each outlet arm member 318 may be profiled on the proximal portion thereof corresponding to the blade(s) of the impeller 202.

Any number of inlet arm members 312 may define any number of shroud sub-inlets 316 therebetween, and any number of outlet arm members 318 may define any number of pump sub-outlets 320 therebetween.

It will be appreciated that the shroud inlet 308, the pump unit inlet, and the sub-inlets thereof may be outlets, and the shroud outlet 310, the pump unit outlet, and the sub-outlets thereof may be inlets, depending on the rotational direction of the impeller 202.

Referring back to FIG. 2, the electrical driveline 206 optionally includes a first driveline portion 212 extending between the guidewire 208 and the second contact element 110, and a second driveline portion 214 extending between the second contact element 110 and along the shroud 200, according to at least one embodiment. The first driveline portion 212 is electrically connectable to the second driveline portion 214.

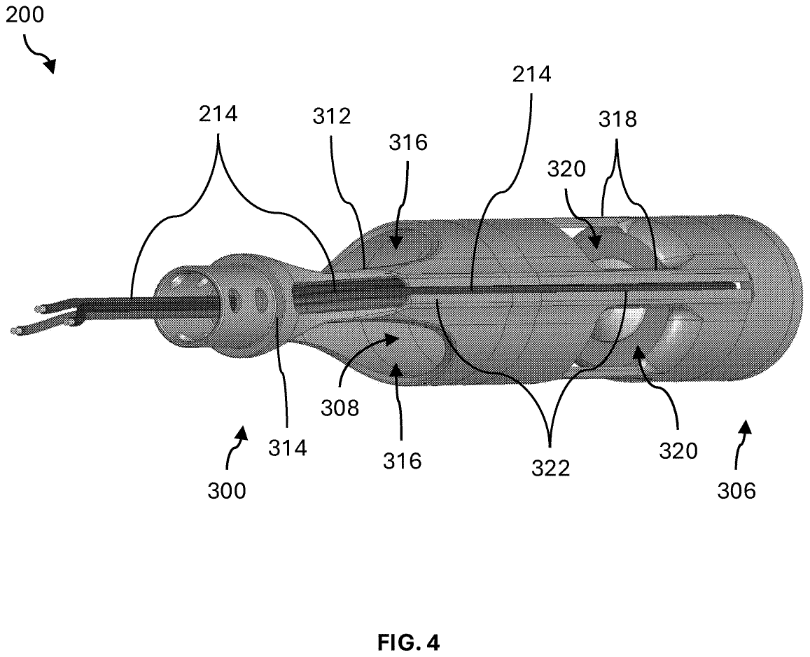

As illustrated in FIGS. 3-4, the second driveline portion 214, illustrated as three electrical cables, extends between the first and second shroud end portions 300, 3006 along an outer surface of the shroud wall (also referred to herein as a “pump unit wall”) and is electrically connected to the first motor end portion 302, according to at least one embodiment. The second driveline portion 214 may extend along a full or partial longitudinal length of the shroud 200. The second driveline portion 214 extends proximally away from the first shroud end portion 300 at the junction body 314.

The second driveline portion 214 may extend along an inner surface of the shroud wall or within the boundaries of the shroud wall. The second driveline portion 214 may also extend along an outer surface, an inner surface, or within the boundaries of the motor wall (also referred to herein as a “drive unit wall”). The second driveline portion 214 may extend within the impeller 202, for example along an impeller rotation axis defined by the impeller 202.

Alternatively, the second driveline portion 214 may extend between the first and second motor end portions 302, 304 and be electrically connected to the first motor end portion 302 or to both the first and second motor end portions 302, 304. The second driveline portion 214 may extend along a full or partial longitudinal length of the electric motor 204.

Alternatively, the second driveline portion 214 may extend between the first shroud end portion 300 and the second motor end portion 304 and be electrically connected to the first motor end portion 302 or to both the first and second motor end portions 302, 304. The second driveline portion 214 may extend along a full longitudinal length of the electric motor 204 and along a full or partial longitudinal length of the shroud 200.

Still referring to FIGS. 3-4, the shroud 200 optionally includes a guide 322, which may include, for example, a recess, a channel, a groove, or a track extending along an outer surface of the shroud wall, including along one of the inlet arm members 312, one of the outlet arm members 318, and the junction body 314, according to at least one embodiment. The guide 322 may be integrally formed with the shroud wall; alternatively, the guide 322 may be a separate component coupled to the shroud wall.

The guide 322 is sized and shaped to receive the second driveline portion 214 therein. For example, the guide 322 may define a cavity portion and an opening portion communicating with the cavity portion, the opening portion having a transverse dimension smaller than a corresponding transverse dimension of the cavity portion, such that only one of the electrical cables can pass through the opening portion at a time to be disposed within the cavity portion.

Alternatively, the guide 322 may be disposed along an inner surface of the shroud wall or within the boundaries of the shroud wall. The guide 322 may also be disposed along an outer surface, an inner surface, or within the boundaries of the motor wall.

The guide 322 advantageously prevents or mitigates the risk of thrombogenesis associated with an inappropriate surface finish of the second driveline portion 214 when exposed to blood by isolating or confining the second driveline portion 214 from blood contact. The guide 322 may also advantageously facilitate routing of the second driveline portion 214 along the shroud wall, particularly given the small size of the second driveline portion 214 and the difficulty of manipulating it.

It will be appreciated that the second driveline portion 214 may extend along the shroud 200 and/or the electric motor 204 directly on the shroud wall and/or the motor wall without requiring the guide 322.

Still referring to FIGS. 3-4, the second driveline portion 214 extending along the shroud wall, as described herein, is coupled to the shroud wall and/or to the guide 322, according to at least one embodiment. Accordingly, the second driveline portion 214 may be coupled along a full or partial longitudinal length of the shroud 200. In particular, the second driveline portion 214 may be mechanically coupled to the shroud wall and, depending on the coupling, may be maintained in physical contact therewith. For example, the second driveline portion 214 may be linked, connected, secured, fastened, fixedly attached, bonded, glued, welded, soldered, overmolded, integrated, or embedded with or within the shroud wall. The second driveline portion 214 may be embedded within the shroud wall. When disposed along the motor wall, the second driveline portion 214 may be coupled to the motor wall in a manner similar to its coupling to the shroud wall.

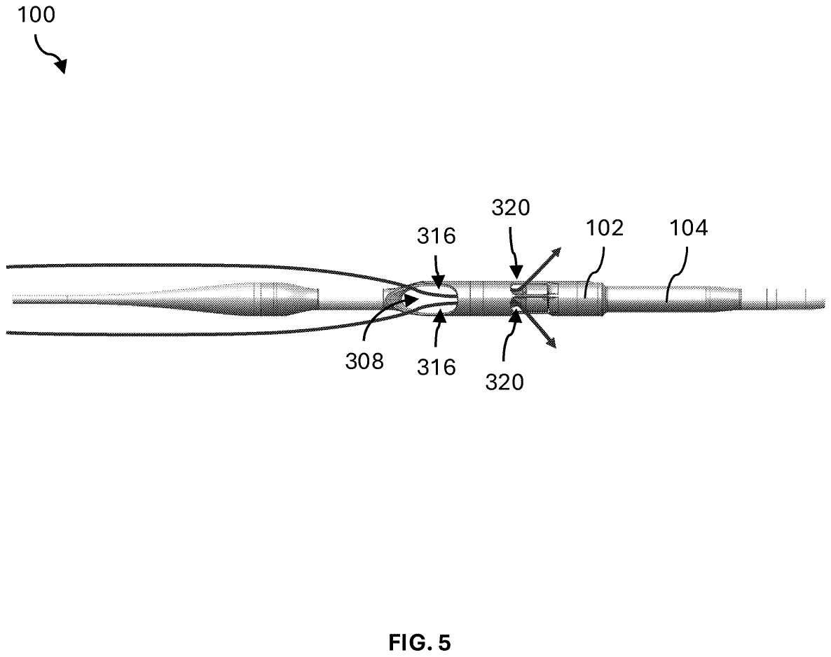

FIG. 5 illustrates blood flow (represented by arrows in FIG. 5) moving from the two shroud sub-inlets 316 to the shroud inlet 308, into the shroud internal passage (not visible in FIG. 5), and to the five shroud sub-outlets 320, according to at least one embodiment. The shroud inlet 308 (also referred to herein as an “axial shroud inlet” or a “longitudinal shroud inlet”) defines an inlet opening oriented toward and facing an upstream blood flow. The inlet opening is substantially centered about the impeller rotation axis and directs blood flow axially, relative to the impeller rotation axis, from the two shroud sub-inlets 316 into the shroud internal passage.

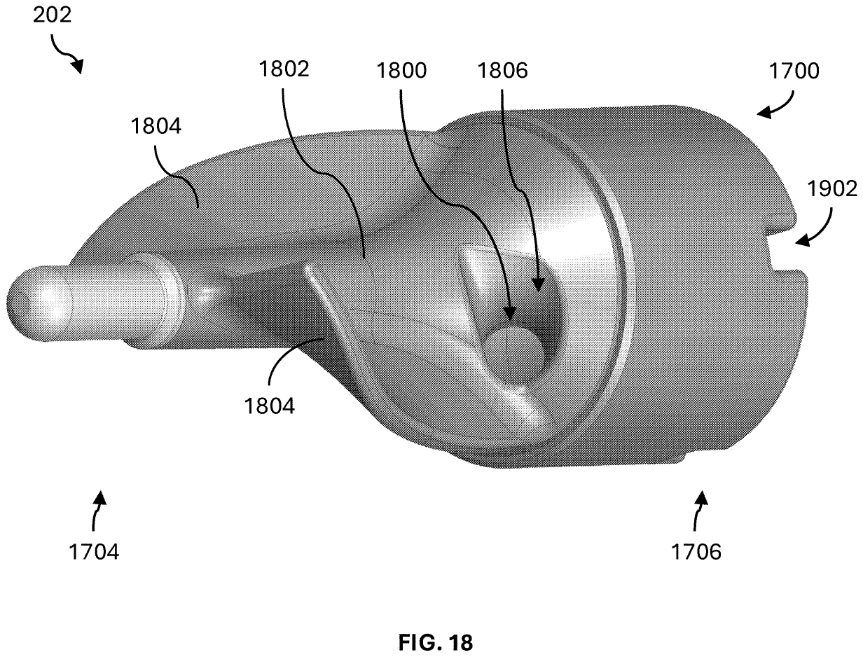

The shroud sub-outlets 320 (also referred to herein as “radial shroud sub-outlets” or “transverse shroud sub-outlets”) define openings oriented radially relative to the impeller rotation axis. A downstream taper surface of the impeller 202 (best illustrated in FIG. 18, 20, for example) is oriented obliquely relative to the impeller rotation axis and redirects at least a portion of the blood flow radially outward relative to the impeller rotation axis through the shroud sub-outlets 320.

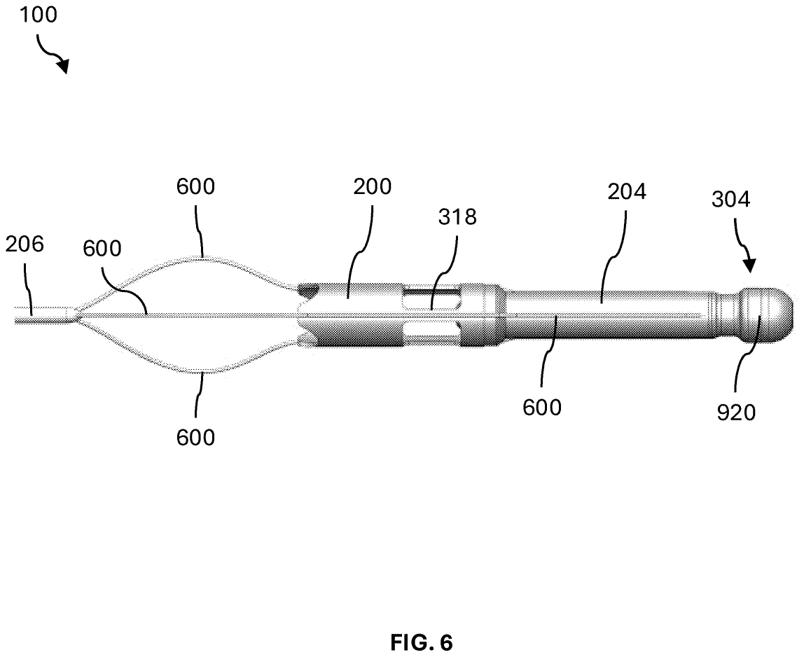

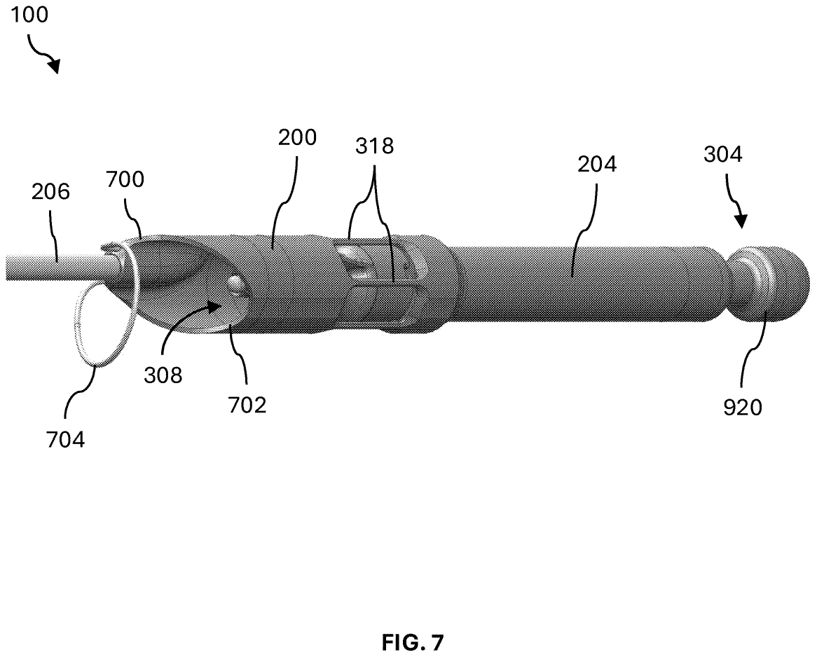

FIGS. 6, 7 illustrate the transcatheter implantable blood pump 100 without the optional two inlet arm members 312, according to some embodiments.

In FIG. 6, the electrical driveline 206 splits into three driveline components 600, according to at least one embodiment. Each driveline component 600 is radially outwardly curved relative to the impeller rotation axis and extends proximally from the first shroud end portion 300 at a circumferential portion of the shroud wall. At least one the driveline components 600 extends along an outlet arm members 318. Each driveline component may include the first driveline portion 212 and/or the second driveline portion 214.

In FIG. 7, the first driveline portion 212 extends proximally away from a circumferential portion of the shroud wall, according to at least one embodiment. The shroud wall optionally defines a shroud inlet 308 with an atraumatic beveled profile including a proximal bevel portion 700 and a distal bevel portion 702. An electrical connection between the first driveline portion 212 and the second driveline portion 214 (not visible in FIG. 7) may be disposed at the proximal bevel portion 700. The first shroud end portion 300 may include a projection 704, such as a looped ring projecting from the proximal bevel portion 700, preventing or mitigating suction-induced lumen wall apposition at the shroud inlet 308 when the transcatheter implantable blood pump 100 is implanted within a subject's lumen.

The shroud 200 may include a flexible tubular body (not shown), such as a cannula-like structure, disposed at the first shroud end portion 300 and extending proximally therefrom. The flexible tubular body defines a tube inner passage in fluid communication with the shroud internal passage. The second driveline portion 214 may extend along the flexible tubular body, for example within a guide 322, and may be coupled thereto, as described herein with respect to the shroud 200.

The flexible tubular body is more flexible than device body 118 formed by the shroud 200 and the electric motor 204.

The flexible tube advantageously increases the distance over which blood may be moved within a subject's body, as compared to the shroud 200 alone, while preserving the ability of the transcatheter implantable blood pump 100 to be advanced through tortuous anatomy due to the capacity of the flexible tube to conform thereto. The transcatheter implantable blood pump 100 provided with the flexible tube may be particularly useful when blood needs to be moved upstream and/or downstream of an acute bend in a subject's lumen beyond which the device body 118 cannot be advanced or implanted, but through which the flexible tube can extend. Such a configuration may be advantageous, for example, when the transcatheter implantable blood pump 100 is implanted in a transvalvular position to move blood from the left ventricle into the aorta.

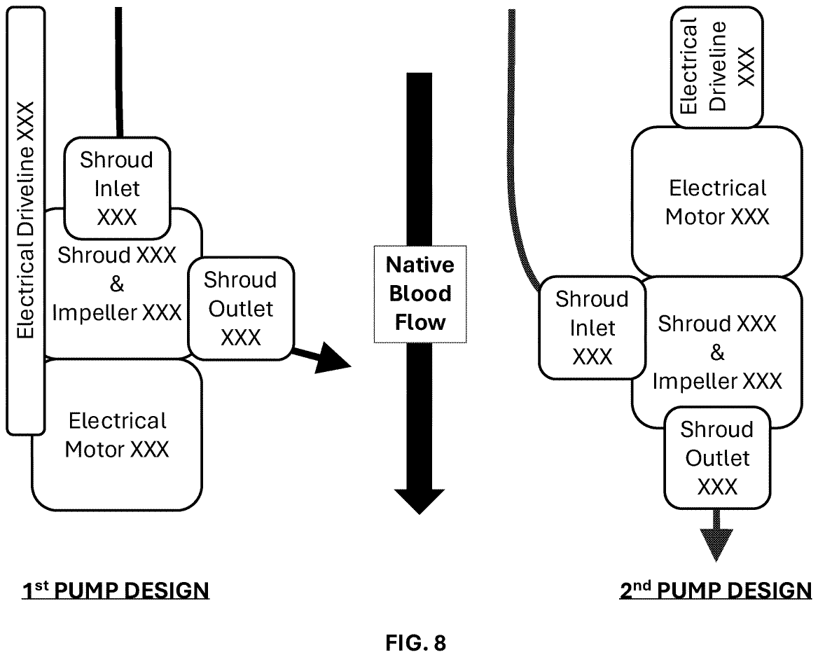

FIG. 8 schematically represents a first pump design corresponding to the transcatheter implantable blood pump 100 and a second pump design corresponding to the transcatheter implantable blood pump 100, but with the electrical driveline not extending along the shroud 200 and/or the electric motor 204 and instead extending distally away from the second motor end portion 304. In the second pump design, the impeller 202 is rotated in an opposite rotational direction relative to the first pump design. Unless otherwise indicated herein, the transcatheter implantable blood pump 100 of the first pump design 1 and the second pump design 2 are identical and were tested under the same experimental conditions for comparison purposes. Native blood flow direction within a subject's cardiovascular system is indicated by a bold black arrow in FIG. 8.

In the first pump design, the shroud inlet 308 defines an inlet opening oriented toward and facing upstream blood flow and is substantially centered about the impeller rotation axis, as described herein. Accordingly, the shroud inlet 308 may be characterized as an axial shroud inlet or a longitudinal shroud inlet. The shroud sub-outlets 320 defines openings oriented radially relative to the impeller rotation axis. Accordingly, the shroud sub-outlets 320 may be characterized as radial shroud sub-outlets or transverse shroud sub-outlets.

In the second pump design, the transcatheter implantable blood pump 100 includes five shroud sub-inlets 316 defining inlet openings oriented radially relative to the impeller rotation axis. Accordingly, the shroud sub-inlets 316 may be characterized as radial shroud sub-inlets or transverse shroud sub-inlets. The shroud outlet 310 defines an outlet opening oriented toward downstream blood flow and is substantially centered about the impeller rotation axis. Accordingly, the shroud outlet 310 may be characterized as an axial shroud outlet or a longitudinal shroud outlet.

Graph 1 below compares experimental fluid outflow data for the first and second pump designs as a function of impeller rotational speed.

As demonstrated by Graph 1, the first pump design exhibits higher fluid outflow than the second pump design across impeller rotational speeds ranging from approximately 10,000 rpm to 30,000 rpm. In particular, the first pump design generally provides approximately a 1.5-fold to 2.0-fold increase in fluid outflow relative to the second pump design across rotational speeds ranging from approximately 10,000 rpm to 30,000 rpm. Similar experimental results are obtained for impeller rotational speeds between 30,000 rpm and approximately 50,000 rpm. The observed performance difference may be attributable, at least in part, to the differing orientation of the shroud inlet between the first pump design and the second pump design.

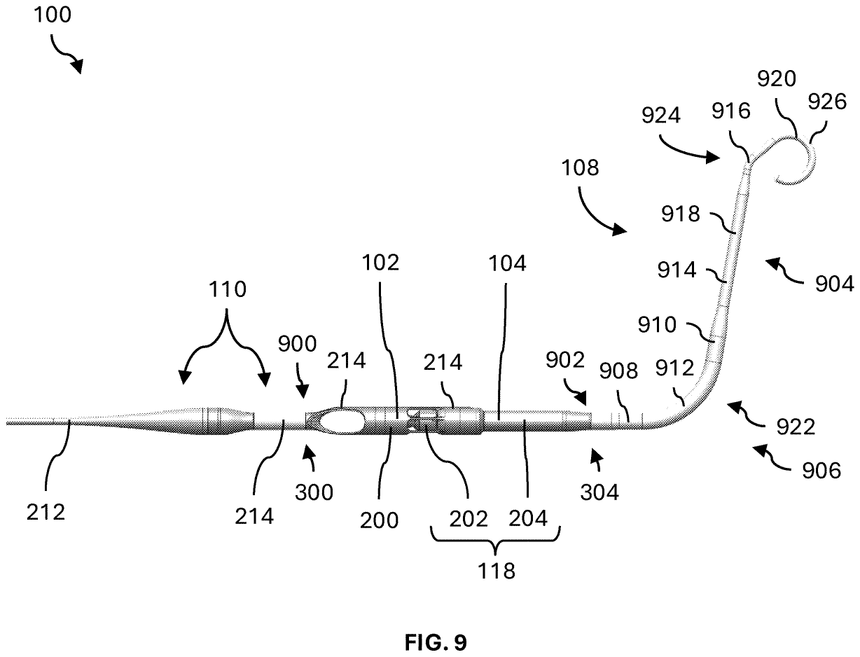

FIG. 9 illustrates the transcatheter implantable blood pump 100 including the first contact element 108 extending distally from the second motor end portion 304, and the second contact element 110 extending proximally from the first shroud end portion 300, according to at least one embodiment. The device body 118 formed by the pump unit 102 and the drive unit 104 have a first body end portion 900 and a second body end portion 902. The first body end portion 900 corresponds to the first pump unit end portion and the first shroud end portion 300. The second body end portion 902 corresponds to the second drive end unit and the second motor end portion 304.

The first contact element 108 includes at least two atraumatic contact portions configured to atraumatically contact a lumen wall of a subject's body. The first contact element 108 is transitionable between (i) a delivery configuration for advancing the transcatheter implantable blood pump 100 within the subject's lumen, and (ii) a contact configuration in which the at least two atraumatic contact portions atraumatically contact the subject's lumen wall within the subject's lumen.

As illustrated in FIG. 9, the first contact element 108 includes a terminal portion 904 and a deflection portion 906 extending between second body end portion 902 and the terminal portion 904, according to at least one embodiment. The deflection portion 906 includes a first segment 908 extending distally, and optionally coaxially, from the second body end portion 902; a second segment 910; and a first curved segment 912 disposed therebetween. The deflection portion 906 optionally includes at least one atraumatic contact portion.

The terminal portion 904 includes a first segment 914 extending from the second segment 910 of the deflection portion 906; a second curved segment 916; and an intermediate segment 918 disposed therebetween. The terminal portion 904 optionally includes at least one atraumatic contact portion.

The first contact element 108 optionally includes a first capturable element 920 extending from the second segment 916 of the terminal portion 904 and configured to be captured by a retrieval instrument within a subject's lumen, as described herein.

Still referring to FIG. 9, the deflection portion 906 includes a first atraumatic contact portion 922, and the terminal portion 904 a second atraumatic contact portion 924, totaling of two atraumatic contact portions, according to at least one embodiment.

In particular, the first atraumatic contact portion 922 is optionally provided by the first curved segment 912 of the deflection portion 906, and the second atraumatic contact portion 924 is optionally provided by the second curved segment 916 (also referred to herein as a “transition segment”) of the terminal portion 904. In the absence of the optional first capturable element 920, the second curved segment 916 may instead be defined by a terminal segment, such that the second atraumatic contact portion 924 is optionally provided by the terminal segment. The first curved segment 912 and the second curved segment 916, or alternatively the terminal segment, are spaced apart from one another along a longitudinal length of the first contact element 108.

Alternatively, the first curved segment 912 and the second curved segment 916, or the terminal segment, may be contiguous with one another along a longitudinal length of the first contact element 108. Similarly, the first and second atraumatic contact portions 922, 924 may be spaced apart from one another or contiguous with one another along the longitudinal length of the first contact element 108.

The atraumatic contact portions, including the first and second atraumatic contact portions 922, 924, are not limited to the first and second curved segments 912, 916, and may include any atraumatic structure, atraumatic geometry, or atraumatic surface configured to contact or engage a subject's lumen wall.

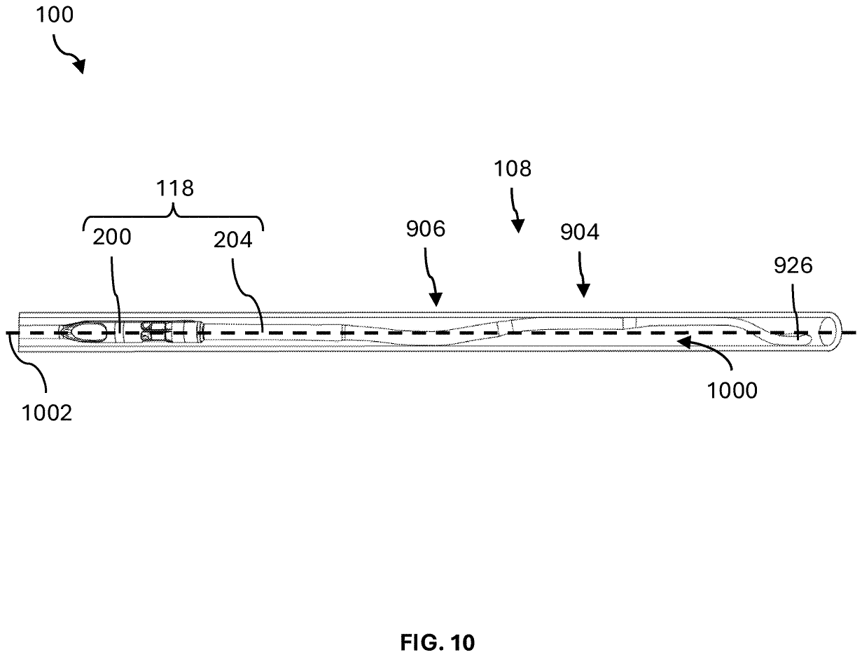

FIG. 10 illustrates the transcatheter implantable blood pump 100 contained within a medical sheath 1000, for example an introducer sheath, according to at least one embodiment. In particular, the first contact element 108 is in a delivery configuration (also referred to herein as a “first linear,” “undeployed,” or “constrained” configuration). In the delivery configuration, the first contact element 108 extends in a substantially linear manner relative to a device body longitudinal axis 1002 defined by the device body 118, the deflection portion 906 is undeflected, and the deflection portion 906 positions the terminal portion 904 in a first terminal portion configuration axially distal relative to the device body 118.

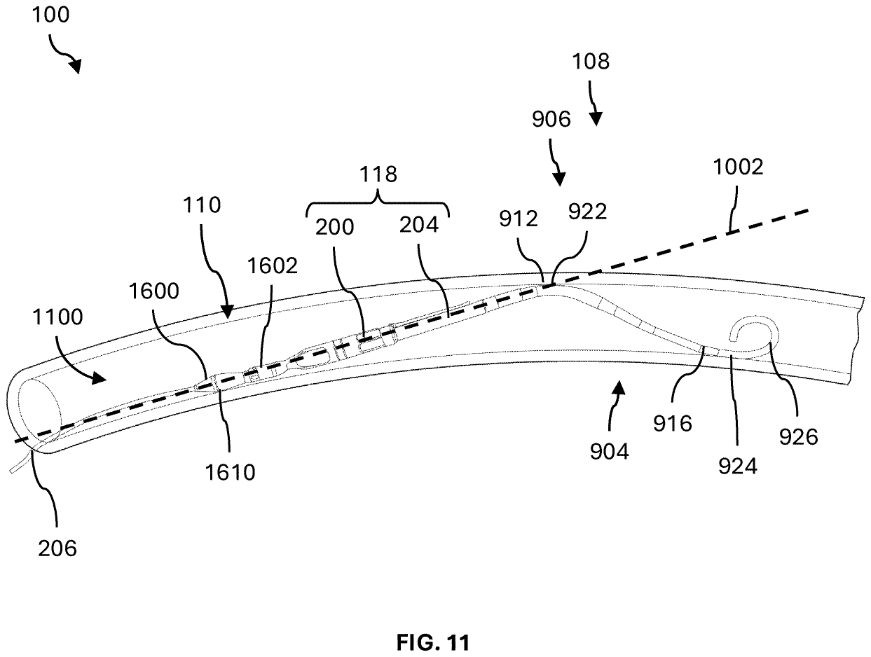

FIG. 11 illustrates the transcatheter implantable blood pump 100 within a subject's lumen 1100, according to at least one embodiment. In particular, first contact element 108 is in a contact configuration (also referred to herein as an “arcuate,” “deployed,” or “unconstrained” configuration). In the contact configuration, the first contact element 108 extends at least partially transverse to the device body longitudinal axis 1002, the deflection portion 906 is deflected, and the deflection portion 906 positions the terminal portion 904 in a second terminal portion configuration radially outward relative to the device body longitudinal axis 1002. The first and second curved segments 912, 916 atraumatically contact the subject's lumen wall at first and second lumen wall regions, respectively.

The first contact element 108 is optionally biased toward the contact configuration from the delivery configuration such that the first and second curved segments 912, 916 exert a radially outward force against and engage the subject's lumen wall at the first and second lumen wall regions, respectively.

The deflection portion 906 is configured to transition the terminal portion 904 between the first and second terminal portion configurations. The first contact element 108 may be biased from the delivery configuration toward the contact configuration.

In the contact configuration, the first contact element 108 optionally defines a substantially planar, two-dimensional (2D) curve-like structure, wherein the first and second curved segments 912, 916 lie within a common 2D plane. The first and second curved segments 912, 916 contact the first and second lumen wall regions, respectively, which are diametrically opposed relative to one another on the subject's lumen wall.

Alternatively, in the contact configuration, the first contact element 108 optionally defines a non-planar, three-dimensional (3D) curve-like structure, wherein the first and second curved segments 912, 916 do not lie within a common 2D plane. The first and second curved segments 912, 916 contact the first and second lumen wall regions, respectively, which are not diametrically opposed relative to one another on the subject's lumen wall.

Whether the first contact element 108 in the contact configuration defines a substantially planar 2D curve-like structure or a non-planar 3D curve-like structure, the first curved segment 912 optionally curves in a first direction, while the second curved segment 916 optionally curves in a second direction different from the first direction. The first direction may be opposite to the second direction.

For comparison with FIG. 11, the first contact element 108 illustrated in FIGS. 2, 9 is in a rest configuration (also referred to herein as a “second arcuate,” “deployed,” or “unconstrained” configuration), according to at least one embodiment. In the rest configuration, the first contact element 108 extends at least partially transverse to the device body longitudinal axis to a greater extent than in the contact configuration. The first contact element 108 is optionally biased toward the rest configuration from the contact configuration and/or from the delivery configuration.

The first contact element 108 defines an increased radial profile in the rest and contact configurations as compared to the delivery configuration.

Referring back to FIG. 9, for example, the first capturable element 920 of the first contact element 108 is optionally provided as a first hook element 926 extending from the second curved segment 916 of the terminal portion 904, according to at least one embodiment. The first hook element 926 is transitionable between (i) a delivery configuration for advancing the transcatheter implantable blood pump 100 within a subject's lumen, and (ii) a capture configuration in which the first hook element 926 is configured to be engaged and captured by a retrieval instrument within the subject's lumen.

The first hook element 926 is optionally biased toward the capture configuration from the delivery configuration. Similarly, the first capturable element 920 may be biased toward the capture configuration from the delivery configurations.

As illustrated in FIG. 10, the first hook element 926 is in the delivery configuration (also referred to herein as a “linear,” “undeployed,” or “constrained” configuration) within the medical sheath 1000, with the first hook element 926 extending in a substantially linear manner relative to the device body longitudinal axis 1002, according to at least one embodiment.

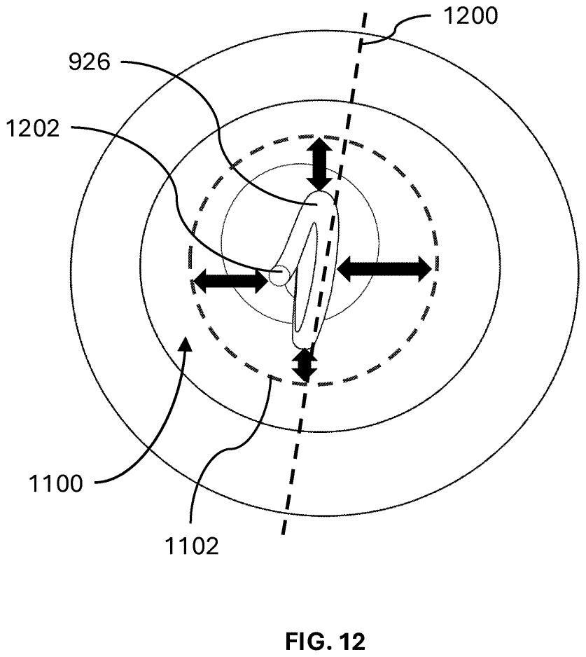

FIG. 12, illustrate the first hook element 926 is in the capture configuration (also referred to herein as an “arcuate,” “deployed,” “unconstrained,” or “rest” configuration) within the subject's lumen 1100, according to at least one embodiment. In the capture configuration, the first hook element 926 extends in a substantially arcuate manner and is positioned within a lumen transverse center region 1102 of the subject's lumen, the lumen transverse center region 1102 being spaced apart from the lumen wall by intraluminal gaps represented by double-headed arrows in FIG. 12. In particular, the second curved segment 916 maintains the first hook element 926 positioned within the lumen transverse center region 1102, spaced apart from the subject's lumen wall.

Positioning of the first hook element 926 within the lumen transverse center region 1102 advantageously facilitates intraluminal capture by a retrieval instrument. For example, when the retrieval instrument is a medical loop snare, the snare may be advanced through the intraluminal gap(s) to encircle the first hook element 926 for capture.

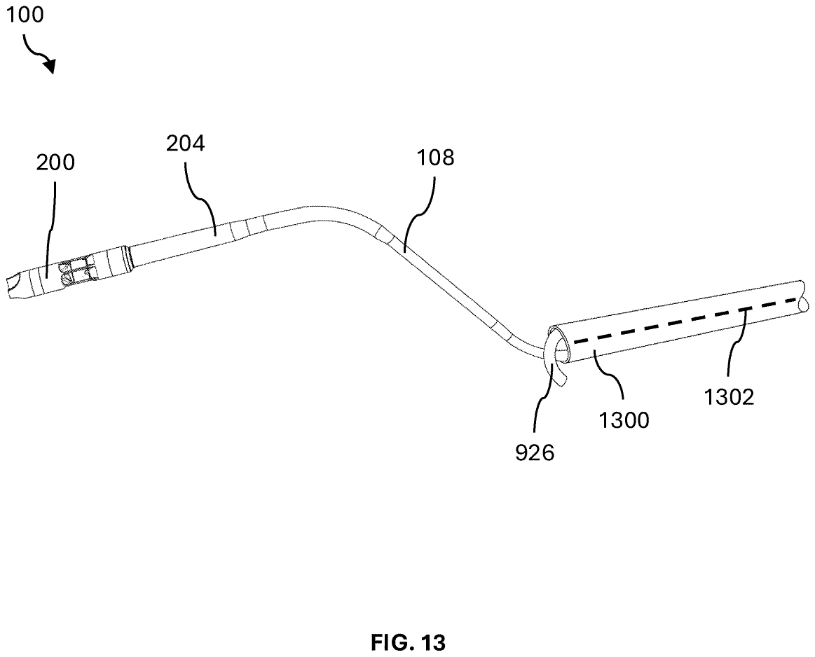

FIG. 13 illustrates the first hook element 926 in a captured configuration (also referred to herein as a “collapsed” or “folded” configuration), according to at least one embodiment. In particular, the first hook element 926 is partially received within a medical sheath 1300 after a retrieval instrument 1302 routed through the medical sheath 1300 and securing the first hook element 926 has been retracted to draw the first hook element 926 at least partially into the medical sheath 1300. The retrieval instrument 1302 is represented by a broken line in FIG. 13 as a way to see thought the medical sheath 1300. In the captured configuration, the first hook element 926 is collapsed or folded upon itself to permit entry into the medical sheath 1300. It will be appreciated that the captured configuration also encompasses instances in which the first hook element 926 is fully contained within the medical sheath 1300.

The first capturable element 920 defines an increased radial profile in the capture configuration as compared to the delivery and captured configurations. The first capturable element 920 further defines an increased radial profile in the captured configuration as compared to the delivery configuration.

As illustrated in FIG. 10, when the first hook element 926 is in the delivery configuration, the first contact element 108 is likewise in the delivery configuration. As best illustrated in FIGS. 2, 9, 11, for example, when the first hook element 926 is in the capture configuration, the first contact element 108 is in either the contact configuration or the rest configuration.

The first hook element 926 is transitionable between any of the delivery, capture, and captured configurations independently of the first contact element 108 being transitioned between any of the delivery, contact, and rest configurations.

Referring to FIGS. 11-12, the first hook element 926 in the capture configuration may be dimensioned such that it is unable to enter a human renal artery lumen, which may be advantageous in the event of intraluminal migration of the transcatheter implantable blood pump 100 implanted within the subject's lumen at an aortic implantation site. Accordingly, in the capture configuration, the first hook element 926 may define a maximum dimension greater than that of the ostial segment of a human renal artery branching from the aorta. The lumen diameter of the ostial segment of human renal arteries in normal adults is typically between approximately four and seven (4-7) millimeters. For example, the first hook element 926 may define a transverse dimension greater than approximately four to seven (4-7) millimeters, as measured within a reference plane 1200 defined by the first hook element 926, depending on the subject's vascular anatomy. In other patient populations, the first hook element 926 may be dimensioned to exceed the renal artery ostial lumen diameter characteristic of that population for the same purpose. The first capturable element 920 may likewise be dimensioned accordingly.

Referring to FIG. 12, the first hook element 926 optionally includes an end tip portion 1202 that lies outside the reference plane 1200, according to at least one embodiment. When abutting the subject's lumen wall, the end tip portion 1202 defines a clearance relative to the subject's lumen wall sufficient to permit a retrieval instrument, such as a medical loop snare, to encircle at least a portion of the first hook element 926 for capture.

The first hook element 926 and the first capturable element 920 may include a magnetic material configured to magnetically couple with a retrieval instrument to facilitate capture, and/or a radiopaque material to enhance fluoroscopic visualization.

Referring back to FIGS. 6, 7, in the absence of the first contact element 108, the first capturable element 920 may instead be provided at the second motor end portion 304, which corresponds to the second body end portion 902. As illustrated, the first capturable element 920 may be provided as a recessed structure defining a circumferential recess configured to be captured by a retrieval instrument within the subject's lumen, for example by being encircled by a medical loop snare.

Referring back to FIG. 5, for example, the drive unit 104 optionally defines a maximum transverse dimension that is smaller than a maximum transverse dimension of the pump unit 102, thereby defining a circumferential space differential along the drive unit 104 relative to the pump unit 102, according to at least one embodiment. The transcatheter implantable blood pump 100 may further include a stent-like contact element (not shown) coupled to the drive unit 104. The stent-like contact element is transitionable between (i) an undeployed configuration for advancing the transcatheter implantable blood pump 100 within a subject's lumen, and (ii) a deployed configuration in which the stent-like contact element is configured to atraumatically contact or engage the subject's lumen wall. In the undeployed configuration, the stent-like contact element, when coupled to the drive unit 104, defines a maximum transverse dimension that is not greater than the maximum transverse dimension of the pump unit 102.

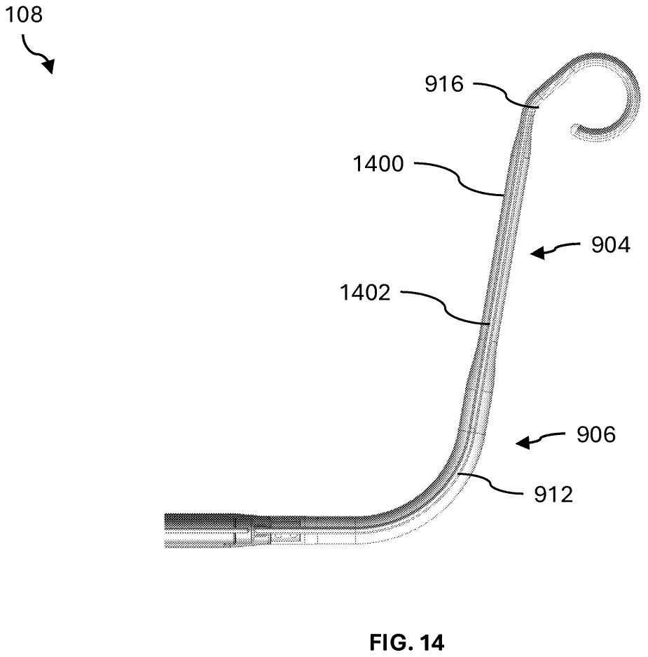

FIG. 14 illustrates the first contact element 108 including a first elongated body 1400 and optionally a second elongated body 1402 extending at least partially along the first elongated body 1400, according to at least one embodiment. The first and/or second elongated bodies 1400, 1402 optionally include the first hook element 926. The second elongated body 1402 is optionally disposed at least partially within the first elongated body 1400; alternatively, the first elongated body 1400 may be disposed at least partially within the second elongated body 1402.

The first contact element 108, including the first and/or second elongated bodies 1400, 1402, optionally define a transverse cross-sectional dimension that varies along a longitudinal length thereof. For example, as illustrated in FIG. 14, the deflection portion 906, or a sub-portion thereof, optionally defines a transverse cross-sectional dimension greater than that of the terminal portion 904, or a sub-portion thereof, according to at least one embodiment.

Alternatively, the terminal portion 904, or a sub-portion thereof, may define a transverse cross-sectional dimension greater than that of the deflection portion 906, or a sub-portion thereof.

For a given transverse cross-sectional dimension of the first contact element 108, the first and second elongated bodies 1400, 1402 may define different transverse cross-sectional dimensions relative to one another.

The flexibility of the first contact element 108, including the first and/or second elongated bodies 1400, 1402, may vary along a longitudinal length thereof. For example, the deflection portion 906, or a sub-portion thereof such as the first curved segment 912, may be less flexible than the terminal portion 904, or a sub-portion thereof such as the second curved segment 916. This may occur, for example, when the deflection portion 906, or a sub-portion thereof, defines a transverse cross-sectional dimension greater than that of the terminal portion 904, or a sub-portion thereof, as illustrated in FIG. 14.

Alternatively, the terminal portion 904, or a sub-portion thereof such as the second curved segment 916, may be less flexible than the deflection portion 906, or a sub-portion thereof such as the first curved segment 912.

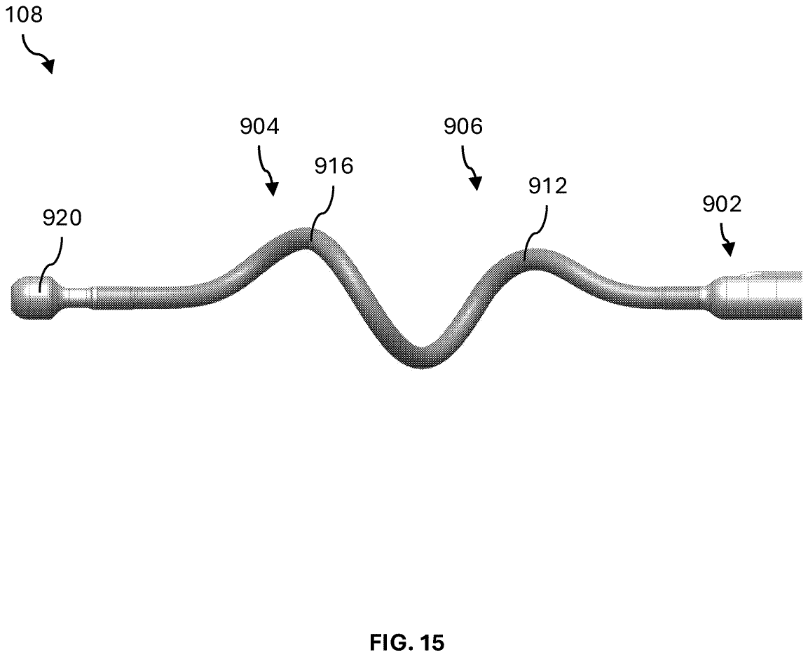

FIG. 15 illustrates the first contact element 108, wherein the first and/or second curved segments 912, 916 optionally define a helical body, according to at least one embodiment. The first capturable element 920 is coupled to the second curved segment 916 of the deflection portion 906 and is as described herein for FIGS. 6-7.

The first and/or second elongated bodies 1400, 1402 may be formed, at least in part, of a biocompatible material and/or a shape-memory material. Biocompatible material may include for example: polyurethane (PU), polyethylene (PE), polypropylene (PP), nylon (polyamide), Pebax® (polyether block amide), polytetrafluoroethylene (PTFE). Shape-memory material may include for example: nitinol and/or stainless steel.

As illustrated in FIG. 14, the second elongated body 1402 optionally includes a wire formed of a shape-memory material and disposed within the first elongated body 1400, which is formed of a biocompatible material that is optionally also a shape-memory material.

The transcatheter implantable blood pump 100 optionally includes at least one of the following features in relation to the first contact element 108:
(i) the device body 118, the shroud 200, and the pump unit 102 are configured for blood flow through a device body inner passage of the device body 118, the shroud internal passage, and the pump unit inner passage, respectively, independently of the first contact element 108;
(ii) blood can flow through the device body inner passage, the shroud internal passage, and the pump unit inner passage defined by device body 118, the shroud 200, and the pump unit 102, respectively, and not by the first contact element 108;
(iii) the first contact element 108 is external to the device body inner passage, the shroud internal passage, and the pump unit inner passage;
(iv) the device body inner passage, the shroud internal passage, and the pump unit inner passage are fully enclosed within the device body 118, the shroud 200, and the pump unit 102, respectively;
(v) the first contact element 108 is structurally separate from and does not surround the device body inner passage, the shroud internal passage, and the pump unit inner passage;
(vi) the first contact element 108 does not circumferentially surround the device body inner passage, the shroud internal passage, and the pump unit inner passage;
(vii) the device body inner passage, the shroud internal passage, and the pump unit inner passage are defined entirely by the device body 118, the shroud 200, and the pump unit 102, respectively, and does not include the first contact element 108;
(viii) the first contact element 108 is configured solely to contact the subject's lumen wall and not to define the device body inner passage, the shroud internal passage, and the pump unit inner passage;
(ix) the first contact element 108 is free of a lumen extending therethrough;
(x) the first contact element 108 defines no internal blood passage in fluid communication with the device body 118, the shroud 200, and the pump unit 102;
(xi) the first contact element 108 is non-tubular; and
(xii) the first contact element 108 is other than a cannula or a sheath.

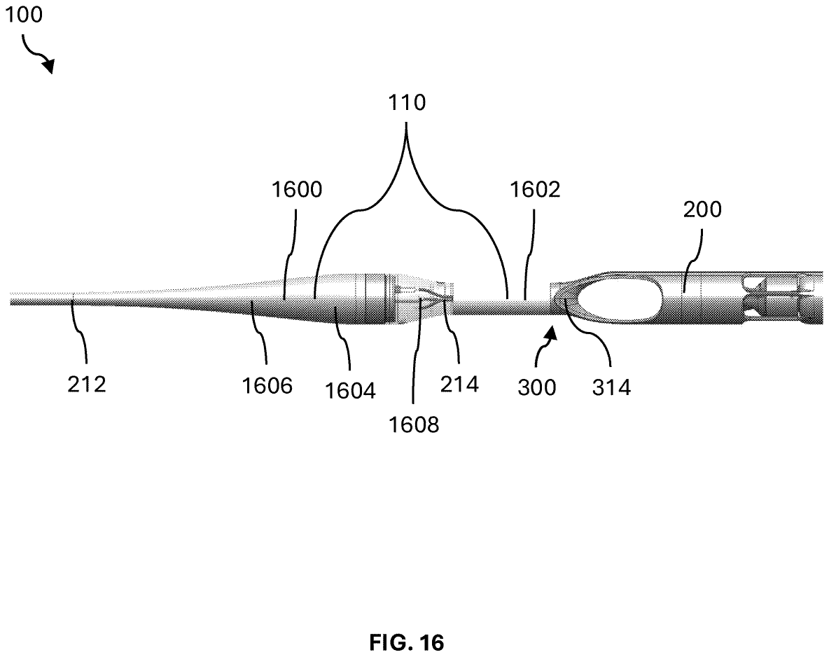

FIG. 16 illustrates the second contact element 110 including at least one atraumatic contact portion configured to atraumatically engage a lumen wall of a subject, according to at least one embodiment.

The second contact element 110 includes a contact portion 1600 and a positioning portion 1602 extending between the first shroud end portion 300, optionally from the junction body 314, and the contact portion 1600. The positioning portion 1602 positions and maintains the contact portion 1600 proximally away from the first shroud end portion 300 and generally coaxial with the device body longitudinal axis.

In particular, the positioning portion 1602 is transitionable between (i) a linear configuration (also referred to herein as a “delivery configuration” for advancing the transcatheter implantable blood pump 100 within the subject's lumen), in which the contact portion 1600 is generally coaxial with the device body longitudinal axis, and (ii) a bent configuration in which the contact portion 1600 is not coaxial with the device body longitudinal axis. The positioning portion 1602 is optionally biased toward the linear configuration from the bent configuration and is optionally less flexible than the driveline 106, in particular the first driveline portion 212.

Alternatively, the first shroud end portion 300 and/or the positioning portion 1602 may be configured to position and maintain the contact portion 1600 non-coaxial with the device body longitudinal axis within a region proximal to the first shroud end portion 300. For example, the positioning portion 1602 may extend non-coaxially from the first shroud end portion 300 and/or may include a curved segment (not shown) configured to position the contact portion 1600 within that region.

Still referring to FIG. 16, the contact portion 1600 optionally includes one atraumatic contact portion 1610 (also referred to herein as a “third atraumatic contact portion”), according to at least one embodiment. In particular, the contact portion 1600 optionally includes a profiled body 1604 having an apex portion 1606 oriented away from the positioning portion 1602. A surface portion of the profiled body 1604 optionally provides the atraumatic contact portion 1600.

Alternatively, or additionally, the at least one atraumatic contact portion 1600 may be provided on a surface of the positioning portion 1602, for example on a longitudinal surface thereof extending at least partially between the first shroud end portion 300 and the contact portion 1600. Each of the contact portion 1600 and the positioning portion 1602 may include a respective atraumatic contact portion 1600, which may be spaced apart from one another or contiguous with one another.

Herein, the at least one atraumatic contact portion of the second contact element 110 is not limited to the surface(s) of the contact and/or the positioning portions 1600, 1602 and may include any atraumatic structure, geometry, or surface configured to contact or engage a lumen wall.

Still referring to FIG. 16, and referring back to FIGS. 2, the second contact element 110 is optionally associated with the electrical driveline 206, according to at least one embodiment. In particular, the positioning portion 1602 optionally defines a channel (not visible in FIGS. 2, 16) configured to receive the electrical driveline 206 and extending between the first shroud end portion 300, optionally from the junction body 314, and the contact portion 1600. The electrical driveline 206 extending along the second contact element 110 may be coupled, as described herein for the shroud 200 and/or the electric motor 204, to at least one of the positioning portion 1602 and the contact portion 1600.

The guide 322 may further extend to the second contact element 110, including the contact and/or the positioning portions 1600, 1602, and function as described herein.

Still referring to FIGS. 2, 16, the second contact element 110 optionally receive and house an electrical connection 1608 between the first driveline portion 212 and the second driveline portion 214. The first driveline portion 212 extends proximally from the contact portion 1600, for example from the apex portion 1606, and the second driveline portion 214 extends distally toward the shroud 200. The second contact element 110 may protect the electrical connection from the external environment. A portion of the contact portion 1600 is represented in transparent in FIG. 16 as a way to see the electrical connection 1608 therethrough.

Referring specifically to FIG. 2, a proximal-most end portion of the driveline 106, or of the first driveline portion 212, includes the electrical connector 210, according to at least one embodiment. The electrical connector 210 is electrically connectable to a console, such as a console configured to be integrated into a garment or mounted to an IV pole, for operating the electric motor 204.

Still referring to FIG. 2, the transcatheter implantable blood pump 100 optionally includes the guidewire 208 extending from the proximal-most end portion of the electrical driveline 206 or the first driveline portion 212, optionally from the electrical connector 210. The guidewire 208 may be configured to increase the anatomical reach of the electrical driveline 206 when advanced within a subject's lumen, as described herein with respect to at least one method.

In particular, depending on the anatomical path, the electrical driveline 206 alone may not be sufficiently long to be advanced through a subject's first access site, along a subject's lumen, and further through a subject's second access site. When coupled to the electrical driveline 206, the guidewire 208 may extend the anatomical reach of the electrical driveline 206 and be advanced through the subject's first access site, along the subject's lumen, and further through the subject's second access site. The guidewire 208 may then be pulled through the subject's second access site to draw the electrical driveline 206 along the anatomical path. The guidewire 208 may subsequently be removed from the electrical driveline 206, for example by severing the guidewire 208 from the electrical driveline 206 or by disconnecting a connector (not shown), which may be integrated to the electrical connector 210, that couples the guidewire 208 to the electrical driveline 206.

Still referring to FIG. 2, the guidewire 208 optionally includes, at a proximal-most portion thereof, a second capturable element 216 optionally provided as a second hook element 218, which may be structurally similar to the first hook element 926. Accordingly, the description of the first hook element 926 applies to the second hook element 218, with any necessary modifications readily appreciable to a person skilled in the art, if applicable. Notably, the second hook element 218 is transitionable between a delivery configuration, a capture configuration, and a captured configuration, and may exhibit biasing characteristics similar to those described for the first hook element 926.

Either of the first and second contact elements 108, 110 may be provided at either the first shroud end portion 300 or the second motor end portion 304.

When provided at the first shroud end portion 300, the first contact element 108 may be associated with the electrical driveline 206, for example by receiving the driveline 106 within a channel defined therein, as described herein with any necessary modifications readily appreciable to a person skilled in the art, if applicable. The first contact element 108 may further be coupled to the driveline 106, as described herein.

When provided at the second motor end portion 304, the first contact element 108 may be independent of, and not associated with, the electrical driveline 206, and may include the first capturable element 920.

Referring back to FIG. 11, the first and second contact elements 108, 110 are configured to orient the device body longitudinal axis of the device body 118 (i.e., the shroud 200 and the electric motor 204) obliquely relative to the subject's lumen 1100, according to at least one embodiment. When implanted in a generally straight subject's lumen defining a longitudinal vascular axis, for example in the lumen of the human descending aorta, the device body longitudinal axis defines an angle θ relative to the longitudinal vascular axis of between 5°-15°, 5°-20°, 5°-25°, 5°-30°, 5°-35°, 5°-40°, or 5°-45°, inclusive.

As illustrated in FIG. 11, the first atraumatic contact portion 922 of the deflection portion 906, the second atraumatic contact portion 924 of the terminal portion 904, and the third atraumatic contact portion 1610 of the contact portion 1600 advantageously stabilize and/or immobilize the transcatheter implantable blood pump 100 within the subject's lumen and advantageously prevent or mitigate vibration thereof.

The contact portion 1600 and the positioning portion 1602, advantageously position and maintain the shroud outlet 310 spaced apart from the subject's lumen wall, thereby improving hydrodynamic performance of the transcatheter implantable blood pump 100.

The device body 118 and the first and second contact elements 108, 110 may define an overall length and/or biasing properties configured to prevent or mitigate migration within a human vascular system from the descending aorta through the aortic arch to an upstream region, such as the ascending aorta. Advantageously, the overall length may be sufficient such that the device body 118 and the first and second contact elements 108, 110 cannot physically traverse the aortic arch. Additionally, or alternatively, the biasing force of the first and/or second contact elements 108, 110 may be sufficient such that the first and/or second contact elements 108, 110 cannot conform to the curvature of the aortic arch. In particular, the first and/or second contact elements 108, 110 may lack sufficient flexibility to pass through the aortic arch.

The first and second contact elements 108, 110 prevent or mitigate excessive tissue overgrowth over or excessive endothelization of them, which is advantageous to atraumatic explant or atraumatic reposition the transcatheter implantable blood pump 100, for example my manipulation of the driveline 106, in particular the second driveline portion 214 externalized from the subject's body, and/or a retrieval instrument capturing the first capturable element 920 and externalized from the subject's body.

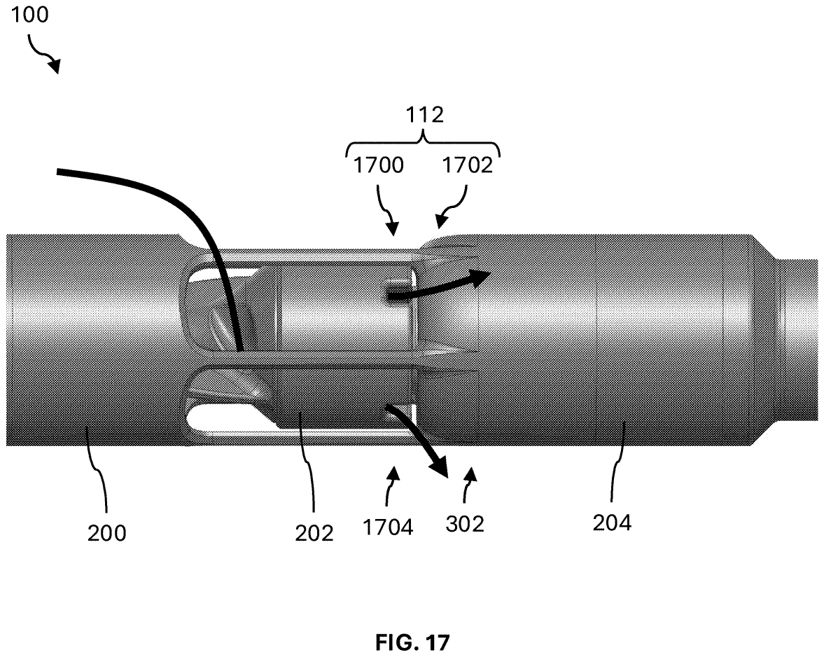

FIG. 17 illustrates the transcatheter implantable blood pump 100 including the first hydrodynamic bearing 112 provided with a first bearing member 1700 associated with the impeller 202 (also referred to herein as a “impeller vaned bearing member 1700” or a “first dynamic bearing member”), and a second bearing member 1702 associated with the electric motor 204 or the drive unit 104 (also referred to herein as a “drive bearing member 1702” or a “first static bearing member”), according to at least one embodiment. The impeller vaned bearing member 1700 is disposed at a second impeller end portion 1706 of the impeller 202, which also has a first impeller end portion 1704 (best illustrated in FIG. 18). The second bearing member 1702 is disposed at the first motor end portion 302.

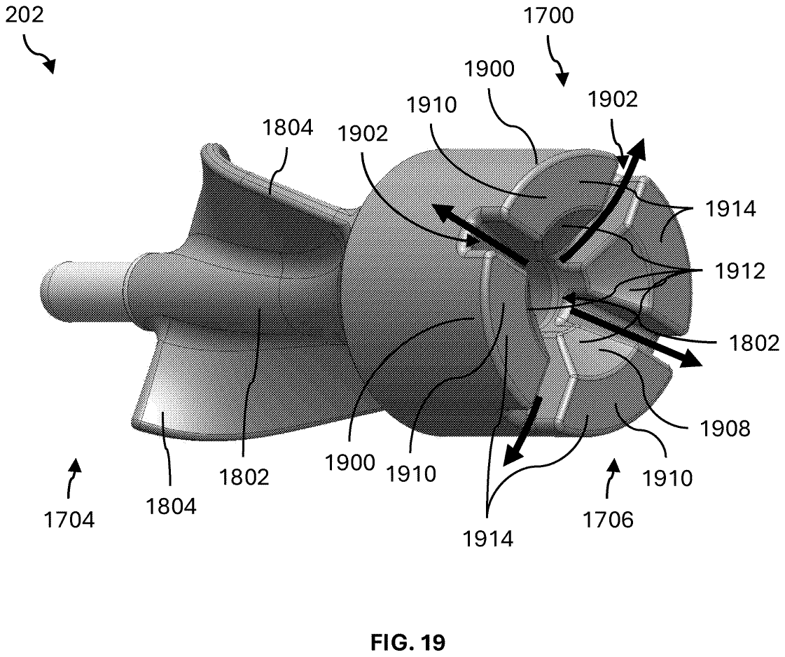

FIGS. 18-19 illustrate the impeller 202 including two impeller hub inlets 1800 (also referred to herein a “port”; only one is visible in FIG. 18) and an impeller hub outlet 1802 (also referred to herein a “port”; visible in FIG. 19) both in fluid communication with an impeller internal passage (not entirely visible in FIGS. 18-19), and the impeller vaned bearing member 1700 including a plurality of vanes 1900 defining a plurality of vane passages 1902 in fluid communication with the impeller hub outlet 1802, according to at least one embodiment.

As best illustrate in FIG. 18, the two impeller hub inlets 1800 are disposed at the base of an impeller hub 1802 between the first and second impeller end portions 1704, 1706, according to at least one embodiment. The impeller hub outlet 1802 is disposed at the second impeller end portion 1706 and is optionally coaxial with the impeller rotation axis. The impeller internal passage extends at least partially within the impeller hub 1802 between the first impeller end portion 1704 and a second impeller end portion 1706. Any number of impeller hub inlets 1800 and impeller hub outlet 1802 may be provided to the impeller 202. The impeller 202 includes at least one impeller blades, for example two impeller blades 1804, extending from the impeller hub 1802 and configured to generate an axial blood inflow.

Still referring to FIG. 18, each inlet of the two impeller hub inlets 1800 optionally include a scooping inlet 1806 (also referred to herein as a “circumference hub inlet” or “port”) defining a respective scoop inlet opening oriented circumferentially upstream relative to a direction of rotation of the impeller, according to at least one embodiment. Each inlet of the two impeller hub inlets 1800 is disposed at a respective pressure-side surface of a corresponding one of the two impeller blades 1804. The scooping inlet 1806 is optionally recessed in the impeller hub 1802; alternatively, the scooping inlet 1806 may protrude from the impeller hub 1802.

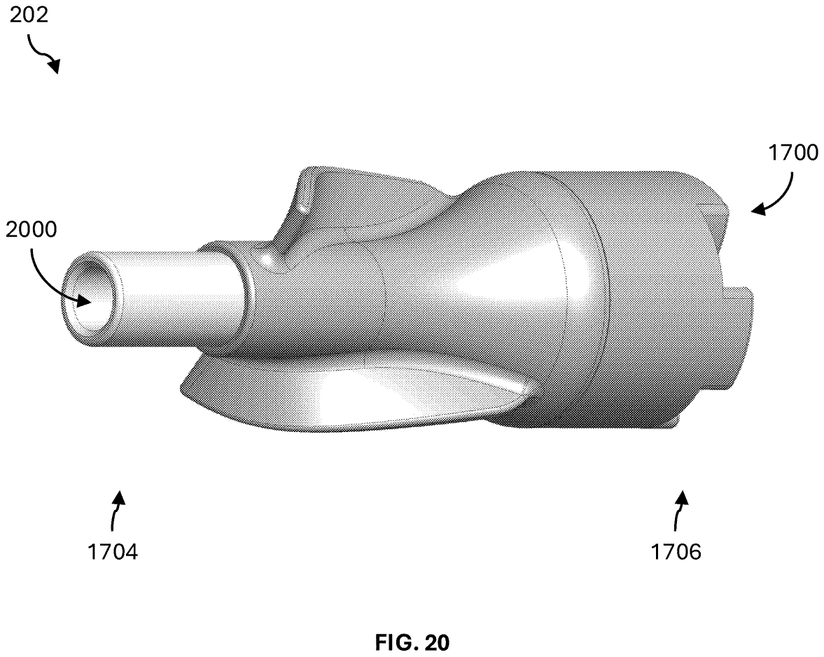

FIG. 20 illustrates the impeller 202 including one impeller hub inlet 2000 disposed at the first impeller end portion 1704 and defining a hub inlet opening oriented toward and facing an upstream blood flow (also referred to herein as an “axial hub inlet” or a “longitudinal hub inlet”), according to at least one embodiment. The hub inlet opening is centered about the impeller rotation axis and directs blood flow axially, relative to the impeller rotation axis, into the impeller internal passage (not entirely visible in FIG. 20), which extends from the first impeller end portion 1704 to the second impeller end portion 1706. The impeller hub inlet 2000 is included in the impeller bearing member 2708, as described herein.

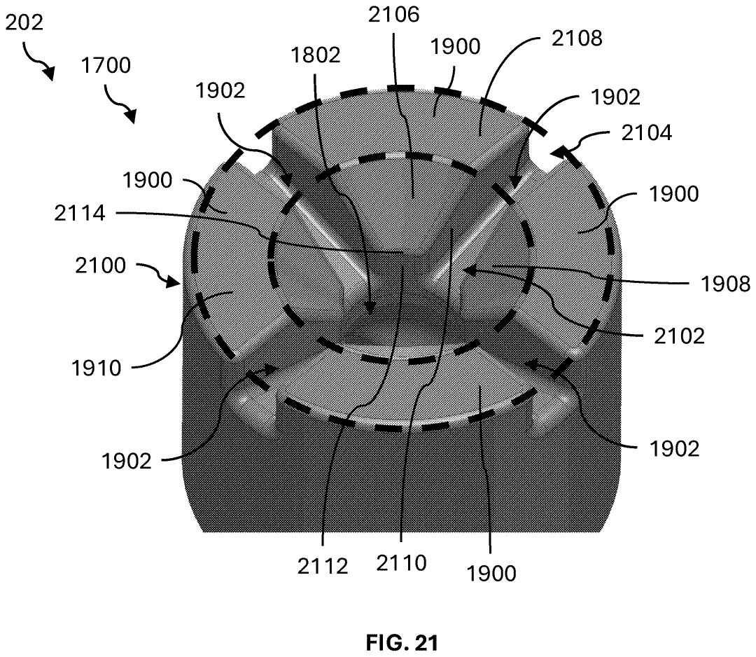

As illustrated in FIG. 21, and referring back to FIGS. 18-19, the plurality of vanes 1900 optionally extends radially outward from the impeller hub outlet 1802 to an outer periphery 2100 of the impeller vaned bearing member 1700, such that the plurality of vanes 1900 extends entirely along the radial length of the impeller vaned bearing member 1700, according to at least one embodiment. As best illustrated in FIG. 21, for example, the plurality of vane passages 1902 defines linear radial paths.

Each passage of the plurality of vane passages 1902 define a central vane end opening 2102 disposed at the impeller hub outlet 1802, or at a central impeller region 2106, and a peripheral vane end opening 2104 disposed at the outer periphery 2100, or the peripheral impeller region 2108. The central impeller region 2106 is represented by the surface defined by the smaller broken-line circle in FIG. 21. The peripheral impeller region 2108 is represented by the surface defined between the smaller-broken line circle and the bigger-broken line circle in FIG. 21. The central vane end opening 2102 may define a transverse dimension smaller or greater than a transverse dimension of the peripheral vane end opening 2104.

Alternatively, the plurality of vanes 1900 may extend radially from a central impeller region 2106 to a peripheral impeller region 2108 of the impeller vaned bearing member 1700, such that plurality of vanes 1900 extends partially along the radial length of the impeller vaned bearing member 1700.

Any number of vanes 1900 may extend radially from the impeller hub outlet 1802 to the outer periphery 2100, or from the central impeller region 2106 to the peripheral impeller region 2108, such that the vanes 1900 may extend entirely or partially along the radial length of the impeller vaned bearing member 1700.

Referring to FIGS. 19, 21, each vane of the plurality of vanes 1900 defines a concave vane surface 1908 (only one concave vane surface is referenced in FIGS. 19, 21 for clarity purpose; also referred to herein as a “first vane surface”) and a flat vane surface 1910 (only one flat vane surface is referenced in FIGS. 19, 21 for clarity purpose; also referred to herein as a “second vane surface”) extending radially outward from the concave vane surface 1908, according to at least one embodiment. The concave vane surface 1908 defines a concave triangular shape, and the flat vane surface 1910 defines a semi-annulus shape.

Referring to FIG. 19, the plurality of concave vane surfaces 1908 of the plurality of vanes 1900 collectively defines a concave impeller surface 1912 (also referred to herein as a “first sub-surface”) to the impeller vaned bearing member 1700, according to at least one embodiment. The concave impeller surface 1912 is a segmented or interrupted or discontinuous surface formed by the plurality of vanes 1900. The concave impeller surface 1912 is optionally disposed at the central impeller region 2106. The first hydrodynamic bearing 112 may function as a hydrodynamic thrust bearing.

Referring to FIG. 21, the plurality of flat vane surfaces 1910 of the plurality of vanes 1900 collectively defines a flat impeller surface 1914 (also referred to herein as a “third sub-surface”) to the impeller vaned bearing member 1700, according to at least one embodiment. The flat impeller surface 1914 is a segmented or interrupted or discontinuous surface formed by the plurality of vanes 1900. The flat impeller surface 1914 is optionally disposed at the peripheral impeller region 2108.

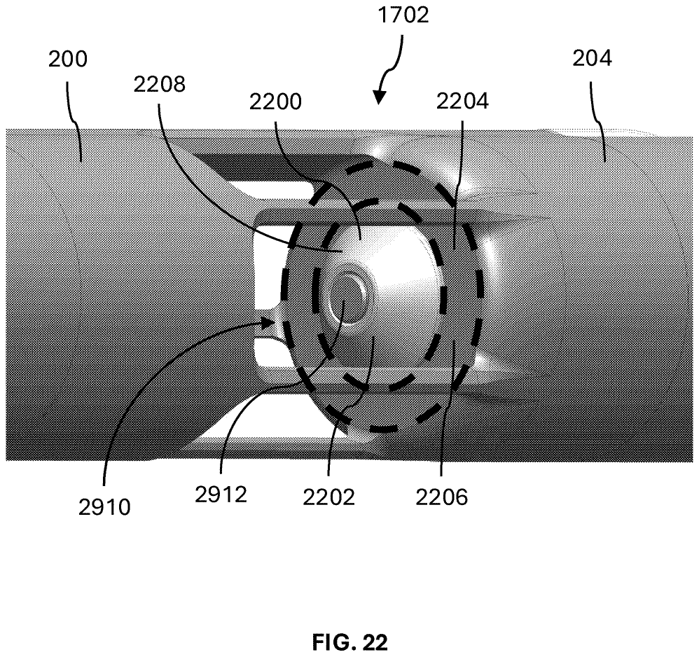

FIG. 22 illustrates the drive bearing member 1702 including a convex drive surface 2200 (also referred to herein as a “second sub-surface”) disposed at a central drive region 2202, and a flat drive surface 2204 (also referred to herein as a “fourth sub-surface”) disposed at a peripheral drive region 2206, according to at least one embodiment. The central drive region 2202 is represented by the surface defined by the smaller broken-line circle in FIG. 22. The peripheral drive region 2206 is represented by the surface defined between the smaller-broken line circle and the bigger-broken line circle in FIG. 22. The central vane end opening 2102 may define a transverse dimension smaller or greater than a transverse dimension of the peripheral vane end opening 2104. Both the convex drive surface 2200 and the flat drive surface 2204 are unsegmented or uninterrupted or continuous surface due to the absence of vane.

A concave/convex bearing interface between the impeller vaned bearing member 1700 and the drive bearing member 1702 advantageously prevents or mitigates wobbling of the impeller 202 under rotation, for example at rotational speed higher than 10,000 rpm.

Alternatively, each first vane surface of the plurality of vanes 1900 may define a convex vane surface, such that the first sub-surface may define a convex bearing surface, and each second vane surface of the plurality of vanes 1900 may define a flat vane surface, such that the third sub-surface may define a flat bearing surface. In this case, the second sub-surface may define a concave bearing surface, and the fourth sub-surface may define a flat bearing surface.

Alternatively, each first vane surface of the plurality of vanes 1900 may define a flat vane surface, such that the first sub-surface may define a flat bearing surface, and each second vane surface of the plurality of vanes 1900 may define a concave vane surface, such that the third sub-surface may define a concave bearing surface. In this case, the second sub-surface may define a flat bearing surface, and the fourth sub-surface may define a convex bearing surface.

Alternatively, each first vane surface of the plurality of vanes 1900 may define a flat vane surface, such that the first sub-surface may define a flat bearing surface, and each second vane surface of the plurality of vanes 1900 may define a convex vane surface, such that the third sub-surface may define a convex bearing surface. In this case, the second sub-surface may define a flat bearing surface, and the fourth sub-surface may define a concave bearing surface.

Referring back to FIG. 21, for example, each vane of the plurality of vanes 1900 includes a peripheral wall 2110 extending from a base surface of the impeller vaned bearing member 1700, and the peripheral wall 2110 includes a wall portion 2112 (also referred to herein as a “spacing wall 2112”) extending parallel and optionally contiguously from the impeller hub outlet 1802 to a surface edge 2114 of the concave vane surface 1908, according to at least one embodiment.

The spacing wall 2112 advantageously enables blood to flow from the impeller internal passage to the plurality of vane passages 1902 when the concave impeller surface 1912 of the impeller vaned bearing member 1700 contacts the convex drive surface 2200 of the drive bearing member 1702, for example prior to rotation of the impeller 202.

Alternatively, the spacing wall 2112 may extend from the base surface at the central impeller region 2106, such that a radial space is defined between the impeller hub outlet 1802 and the spacing wall 2112. The spacing wall 2112 may extend inclined from the base surface, such that a portion of the impeller hub outlet 1802 extending to the surface edge 2114 of the concave vane surface 1908 is oriented toward the outer periphery of the impeller vaned bearing member 1700.

The vane of the plurality of vanes 1900 optionally define, relative to a direction of rotation of the impeller 202, a leading wedge surface and/or a trailing wedge surface. In particular, the leading wedge surface and/or a trailing wedge surface may be defined on the concave vane surface 1908 and/or on the flat vane surface 1910. The leading wedge surface and/or a trailing wedge surface may be defined on any number of the vanes.

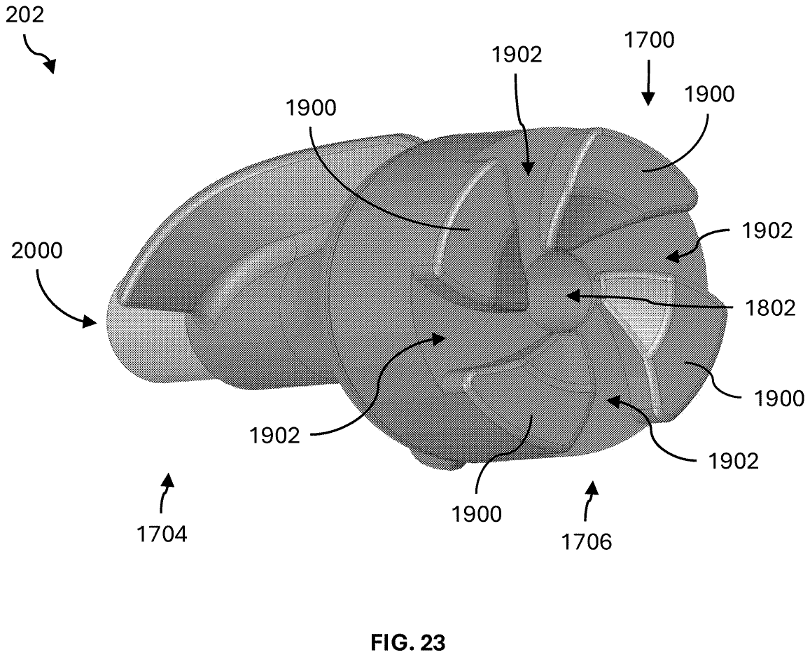

FIG. 23 illustrates the impeller 202 including the impeller vaned bearing member 1700 provided with the plurality of vane passages 1902 defining curved radial paths, the impeller hub inlet 2000 disposed at the first impeller end portion 1704, and the impeller internal passage extending from the impeller hub inlet 2000 to the second impeller end portion 1706, according to at least one embodiment. The impeller 202 illustrated in FIGS. 18-21 may include a plurality of vane passages 1902 defining curved radial paths.

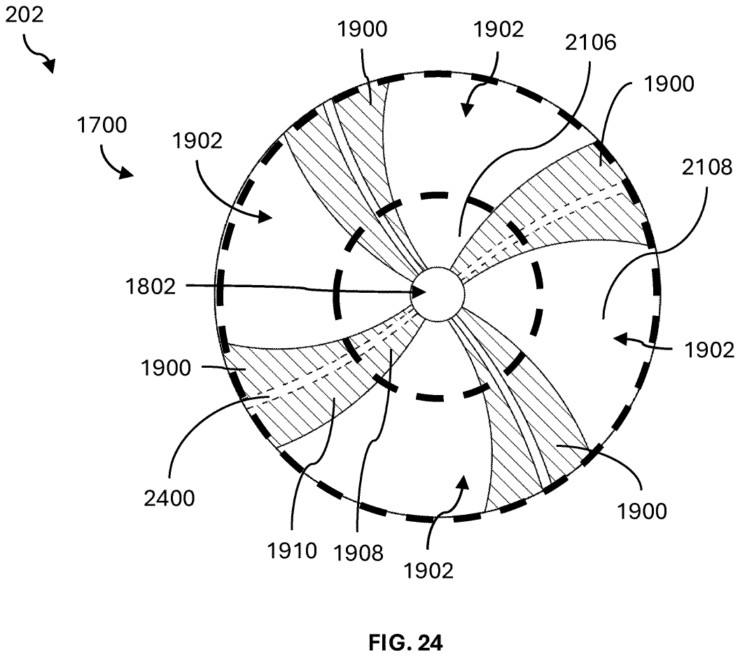

FIG. 24 illustrates the impeller 202 including the impeller vaned bearing member 1700 provided with the plurality of vanes 1900 defining the plurality of plurality of vane passages 1902 and at least one vane sub-passages 2400 defined on a respective concave vane surface 1908 and/or a respective flat vane surface 1910 of a vane 1900, according to at least one embodiment.

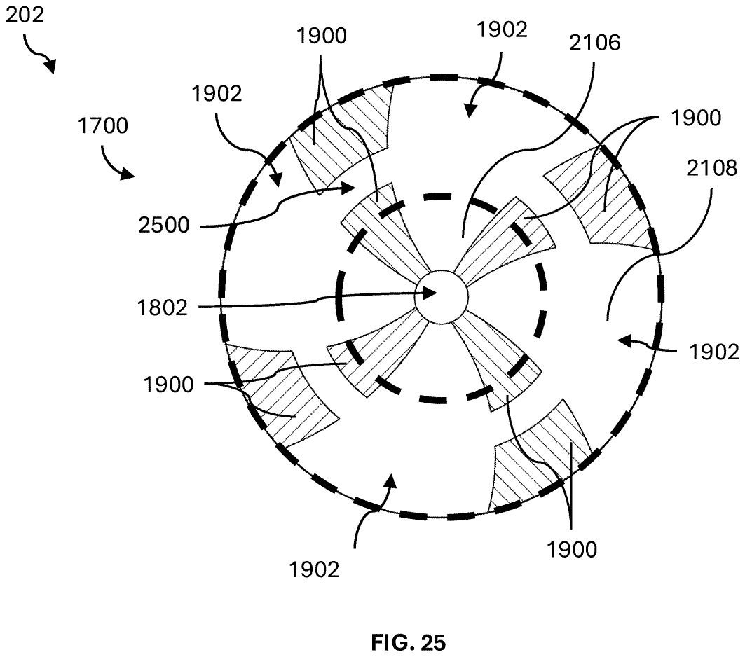

FIG. 25 illustrates the impeller 202 including the impeller vaned bearing member 1700 provided with the plurality of vanes 1900 defining the plurality of plurality of vane passages 1902 and at least one circumference passages 2500, according to at least one embodiment.

The impeller vaned bearing member 1700, including the central impeller region 2106 and/or the peripheral impeller region 2108, may be made of a material having a wear resistance lower or greater than, or equal to, a wear resistance of a material of the drive bearing member 1702, including the central drive region 2202 and/or the peripheral drive region 2206. For example, the central impeller region 2106, may be made of a material, such as titanium, having a wear greater than a material, for example polyether ether ketone (PEEK), of the central drive region 2202.

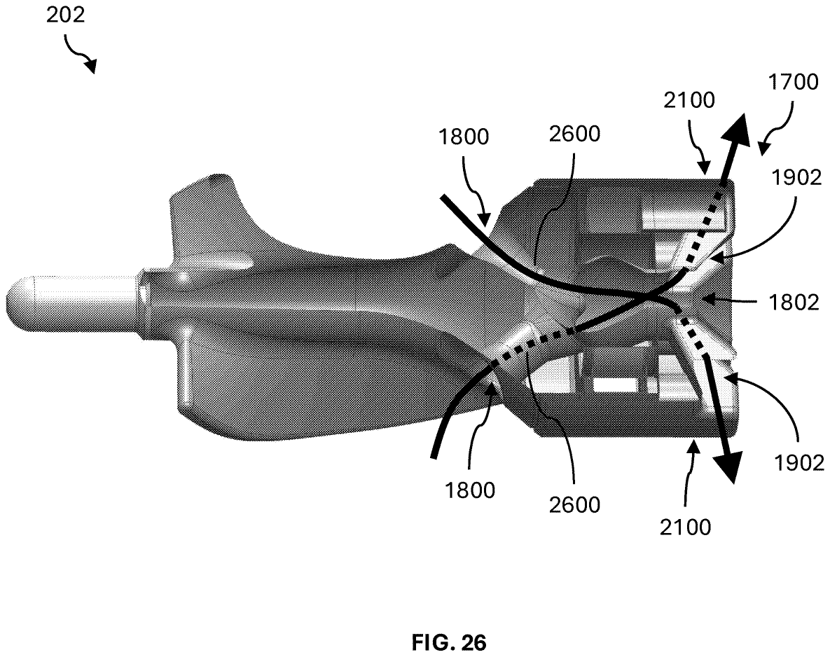

As illustrated in FIG. 26, and referring back to FIGS. 17, 19, a blood flow (represented by arrows in FIGS. 17, 19, 27) generated by rotation of the impeller 202 moves from the two impeller hub inlets 1800, through the impeller internal passage, the impeller hub outlet 1802, and the plurality of vane passages 1902, and to the outer periphery 2100 of the impeller vaned bearing member 1700, according to at least one embodiment. The plurality of vanes 1900 are configured to generate a centrifugal blood outflow.

Referring to FIG. 26, the impeller internal passage includes two internal impeller sub-passages 2600, and each sub-passage 2600 extends from a respective one of the two impeller hub inlets 1800 to the impeller hub outlet 1802, according to at least one embodiment. Each sub-passage 2600 defines a helical path winding about the impeller rotation axis and shaped to facilitate or enhance blood flow therethrough.

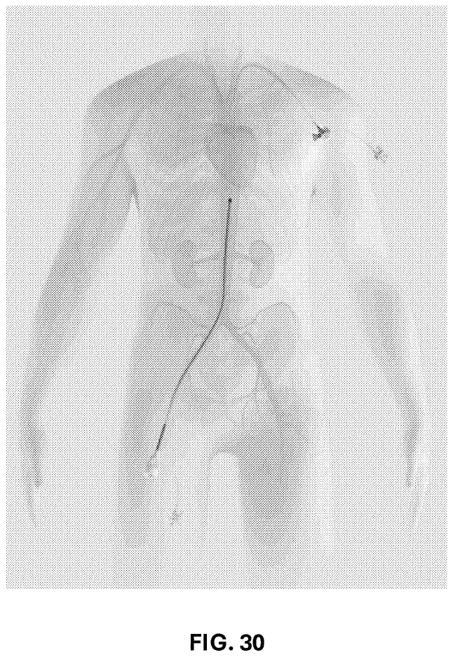

The flat impeller surface 1914 and the flat drive surface 2204 are optionally spaced apart to define an intermediate passage 2700 therebetween, as illustrated by a broken line circle in FIG. 30, through which blood moves radially outward toward the outer periphery 2100, according to at least one embodiment. The intermediate passage 2700 optionally prevents or mitigates contact between the flat impeller surface 1914 and the flat drive surface 2204, for example at startup or during operation. The intermediate passage 2700 is advantageous when the flat impeller surface 1914 and the flat drive surface 2204 are made of materials that wear and/or have inappropriate friction properties when contacting one another.

Rotation of the impeller 202 generates hydrodynamic support between the impeller vaned bearing member 1700 and the drive bearing member 1702. In particular, a pressure-developing fluid film is generated between the concave impeller surface 1912 of the impeller vaned bearing member 1700 and the convex drive surface 2200 of the drive bearing member 1702, and optionally between the flat impeller surface 1914 of the impeller vaned bearing member 1700 and the flat drive surface 2204 of the drive bearing member 1702. At rest, the concave impeller surface 1912 contacts the convex drive surface 2200. The respective materials of the concave impeller surface 1912 and the convex drive surface 2200 may therefore be selected to reduce wear and/or friction at startup or during operation.

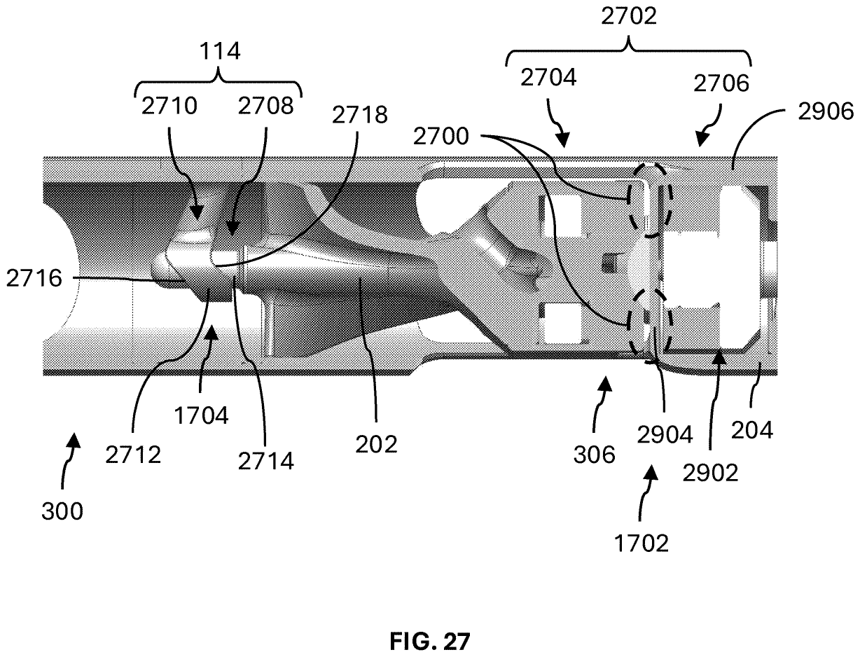

FIG. 27 illustrates a magnetic coupling system 2702 including a first magnetic member 2704 (also referred to herein as an “impeller-coupled magnetic element 2704” or a “driven magnetic element”) associated with the impeller 202, and a second magnetic member 2706 (also referred to herein as an “drive-coupled magnetic element 2706” or a “driving magnetic element”) associated with electric motor 204 or the drive unit 104, according to at least one embodiment. The magnetic coupling system 2702 is configured to magnetically couple the impeller-coupled magnetic element 2704 to the drive-coupled magnetic element 2706 to transmit torque between the electric motor 204 and the impeller 202 for moving blood. The first hydrodynamic bearing 112 is configured to oppose an attractive axial magnetic force between the impeller-coupled magnetic element 2704 and the drive-coupled magnetic element 2706.

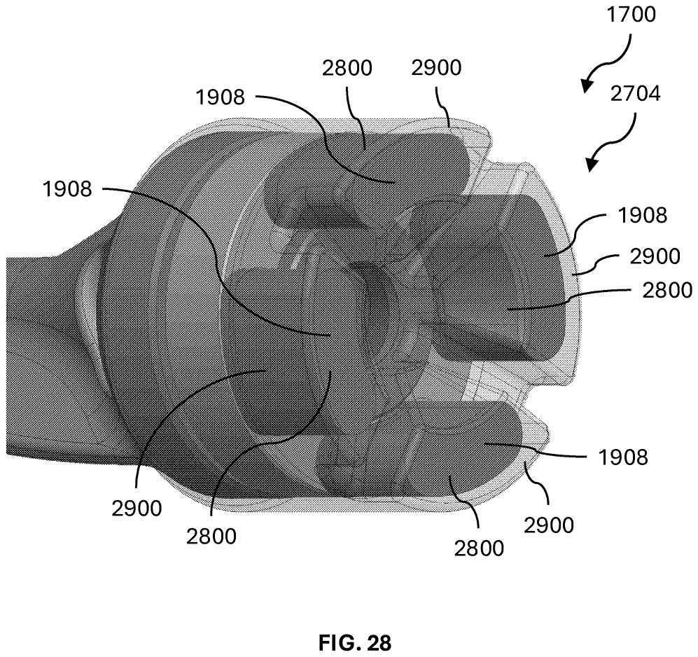

FIG. 28 illustrates the impeller-coupled magnetic element 2704 including a plurality of impeller-coupled magnets 2800, with each magnet 2800 associated with a respective one of the plurality of vanes 1900, according to at least one embodiment. In particular, each magnet 2800 is optionally disposed within a respective one of the plurality of vanes 1900, optionally beneath the flat vane surface 1910 thereof. Alternatively, or additionally, each magnet 2800 may disposed beneath the concave vane surface 1908. Each magnet 2800 may be embedded within a respective one of the plurality of vanes 1900. Each magnet 2800 may form a respective one of the plurality of vanes 1900.

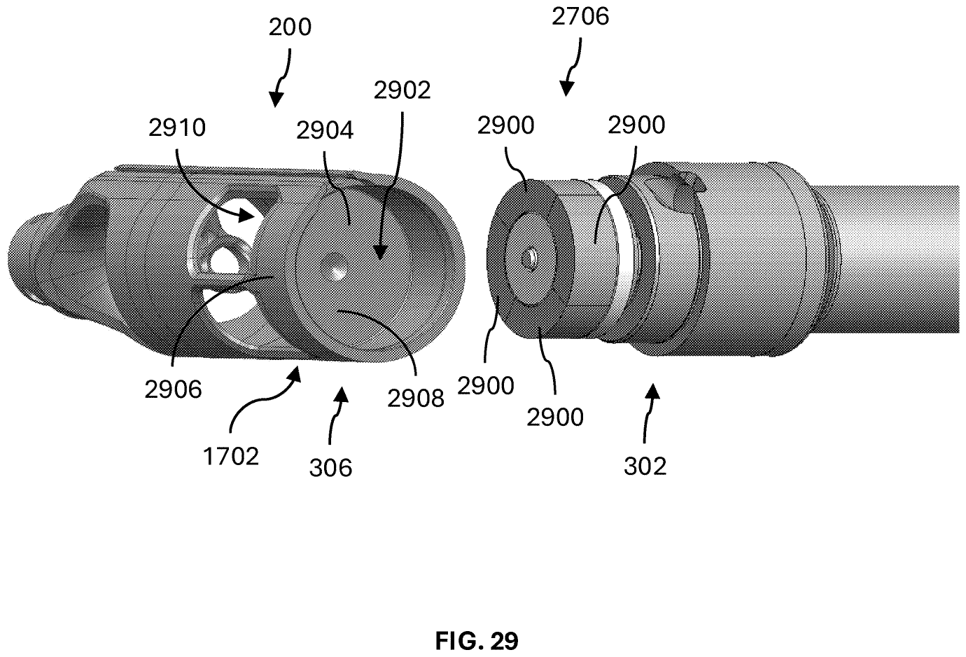

FIG. 29 illustrates the drive-coupled magnetic element 2706 including a plurality of drive-coupled magnets 2900, with each magnet 2900 configured to magnetically couple a respective one of the plurality of impeller-coupled magnets 2800, according to at least one embodiment.

The positioning of the magnets 2800 within the vanes 1900 advantageously improves the attractive magnetic force between the impeller-coupled magnetic element 2704 and the drive-coupled magnetic element 2706, and therefore torque transmission, compared to configurations in which the magnets 2800 are positioned deeper within the impeller-coupled magnetic element 2704 and farther from the concave and flat vane surfaces 1908, 1910.

Referring back to FIGS. 27, 29, the drive-coupled magnetic element 2706 is enclosable into a drive magnetic coupling cavity 2902 disposed at the second shroud end portion 306, according to at least one embodiment. The magnetic coupling cavity 2902 is formed by a separating wall 2904 (also referred to herein as a “bearing wall” or a “first shroud wall”) associated with the drive bearing member 1702, and a tubular wall 2906 (also referred to herein as a “second shroud wall”) extending distally from the separating wall 2904. The tubular wall 2906 is connectable to the first motor end portion 302 to enclose the drive-coupled magnetic element 2706 within the drive magnetic coupling cavity 2902.

The separating wall 2904 includes an inner wall surface 2908 facing the drive-coupled magnetic element 2706, and, as also illustrated in FIG. 22, an outer wall surface 2910 facing the impeller vaned bearing member 1700, according to at least one embodiment. The outer wall surface 2910 defines the flat drive surface 2204 disposed at the peripheral drive region 2206. The outer wall surface 2910 is optionally connected to a conical body 2208 that defines the convex drive surface 2200 disposed at the central drive region 2202. In particular, the outer wall surface 2910 optionally includes a mounting structure, for example a protrusion 2912, to mount the conical body 2208 (also referred to herein as an “axially bored conical body”).

The conical body 2208 is optionally made of a material, for example polyether ether ketone (PEEK), having a wear resistance lower than a wear resistance of a material, for example titanium, of the concave vane surface 1908 and the concave impeller surface 1912 of the impeller vaned bearing member 1700 at the central impeller region 2106. Alternatively, the conical body 2208 is optionally made of a material having a wear resistance greater than a wear resistance of a material of the concave vane surface 1908 and the concave impeller surface 1912 of the impeller vaned bearing member 1700 at the central impeller region 2106.

Referring to FIGS. 27, 29, the drive-coupled magnetic element 2706 is enclosed within the drive magnetic coupling cavity 2902 formed by the tubular wall 2906 (i.e., a single-axial-wall construction). Such configuration advantageously enables a larger drive-coupled magnetic element 2706 to be accommodated for a same maximum transverse dimension, as compared to configurations in which the drive magnetic coupling cavity is defined between the tubular wall 2906 and an additional tubular wall (not shown) of a motor unit (i.e., a dual-axial-wall construction) used to connect the shroud unit to the electric motor 204.

Referring to FIG. 27, the transcatheter implantable blood pump 100 optionally including a second hydrodynamic bearing 114 provided with a first bearing member 2708 associated with the impeller 202 (also referred to herein as a “impeller bearing member 2708”, a “second dynamic bearing member” or a “journal member”), and a second bearing member 2710 associated with the pump unit 102 or the shroud 200 (also referred to herein as a “shroud bearing member 2710”, a “second static bearing member”, or a “sleeve member”), according to at least one embodiment. The second hydrodynamic bearing 114 may function as a hydrodynamic journal bearing.

The impeller bearing member 2708 is disposed at the first impeller end portion 1704, and the shroud bearing member 2710 is disposed at the first shroud end portion 300. The shroud bearing member 2710 includes a tubular body 2712, and the impeller bearing member 2708 includes a tubular body cylindrical body 2714 rotatably receivable in the tubular body 2712. An internal surface of the tubular body 2712 defines a hydrodynamic bearing interface with an external surface of the tubular body cylindrical body 2714.

The tubular body 2712 may define at least one opening (not shown), for example slits, to expose the external surface of the cylindrical body 2714 therethrough. Rotation of the impeller 202 may cause blood to be moved through the at least one opening.

When the impeller 202 is provided with the impeller hub inlet 2000, as illustrated in FIGS. 20, 24, for example, the tubular body cylindrical body 2714 includes an axial hub inlet or a longitudinal hub inlet, as described herein.

The cylindrical body 2714 is optionally made of a material, for example polyether ether ketone (PEEK), having a wear resistance lower than a wear resistance of a material, for example titanium, of the tubular body 2712. Alternatively, the cylindrical body 2714 is optionally made of a material having a wear resistance greater than a wear resistance of a material of the tubular body 2712.

Alternatively, the transcatheter implantable blood pump 100 may include a non-hydrodynamic bearing (not shown) instead of the second hydrodynamic bearing.

Referring to FIG. 27, the tubular body 2712 (also referred to herein as an “obliquely truncated tubular body”) optionally defines a first annular elliptical face 2716 facing proximally away from the impeller 202 and a second annular elliptical face 2718 facing distally toward the impeller 202, according to at least one embodiment. The first and second annular elliptical faces 2716, 2718 are disposed around the impeller rotation axis and are defined by a respective oblique plane (not shown) truncating the tubular body 2712 obliquely relative to the impeller rotation axis. The first and second annular elliptical faces 2716, 2718, and respective oblique planes thereof, are optionally parallel to one another; alternatively, the first and second annular elliptical faces 2716, 2718, and respective oblique planes thereof, may be non-parallel to one another.

Alternatively, the tubular body 2712 may include a right tubular body (not shown) having a first annular face (facing Vs. oriented) proximally away from the impeller 202, and a second annular face (facing Vs. oriented) distally toward the impeller 202, with the first and second annular faces disposed around the impeller rotation axis.

Rotatably received within the obliquely truncated tubular body 2712, the cylindrical body 2714 repeatedly exposes axial portions of its external surface to the internal surface of the obliquely truncated tubular body 2712 and to the surrounding environment, such as blood present within the internal shroud passage, during rotation of the impeller 202. Such repeated exposure of the axial portions of the external surface advantageously reduces or mitigates thrombogenesis by promoting periodic washing of the exposed surfaces with flowing blood and by reducing regions of blood stagnation.

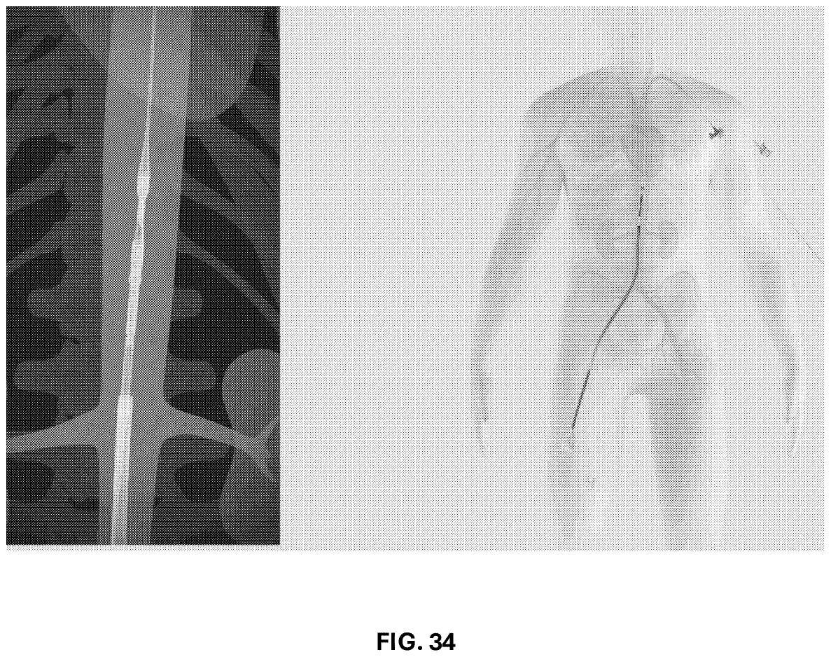

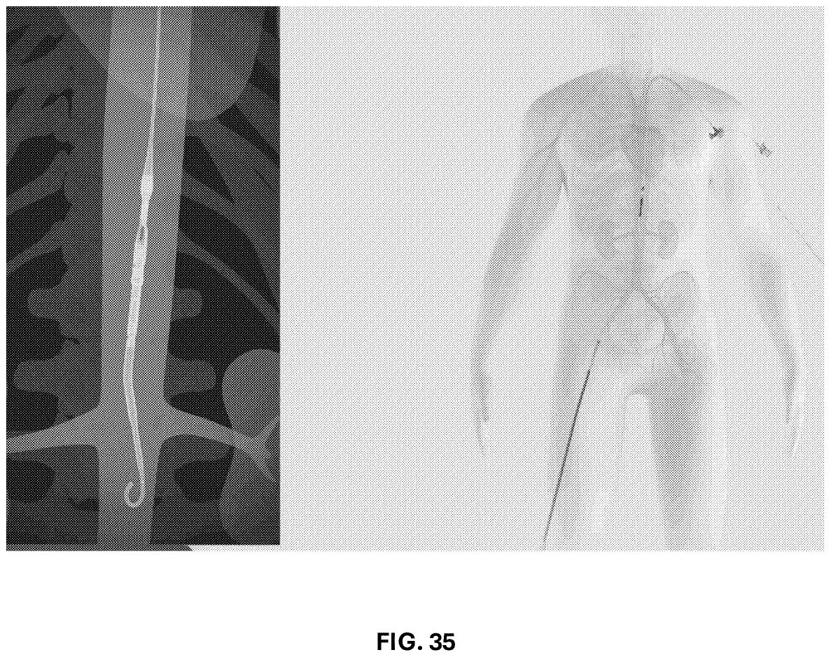

According to another aspect, as schematically represented in FIGS. 30-37, this disclosure relates to a method 3000 of implanting a medical device within a subject's body, according to at least one embodiment. It is noted that reference to the transcatheter implantable blood pump 100, or any other feature described herein, is made for the sole purposes of describing how the method 3000 may be implemented or practiced. Accordingly, such reference is not intended to limit the scope of the method 3000.

FIGS. 30-36 illustrate a sequential overview of an example of how the method 3000 may be performed to implant a medical device within a human aorta. The method 3000 may include the following steps:

Installing a first introducer sheath at a femoral access site and a second introducer sheath at an axillary or subclavian access site of a subject, and advancing the first and second introducer sheaths toward an aortic implantation site or proximate to the aortic implantation site, as illustrated in FIG. 30.

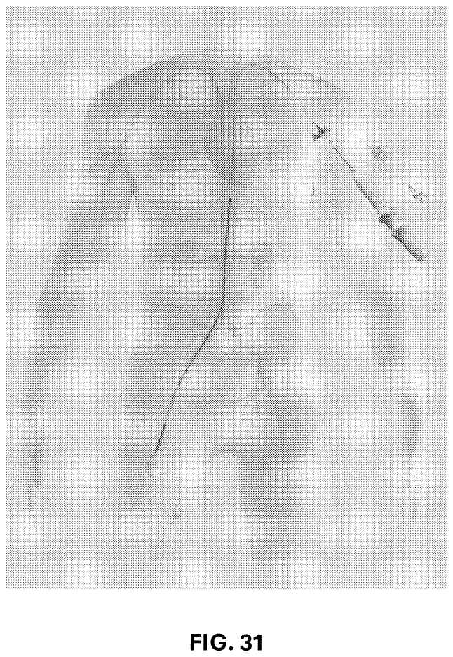

Advancing a medical loop snare through the second introducer sheath to the aortic implantation site or proximate to the aortic implantation site, as illustrated in FIG. 31.

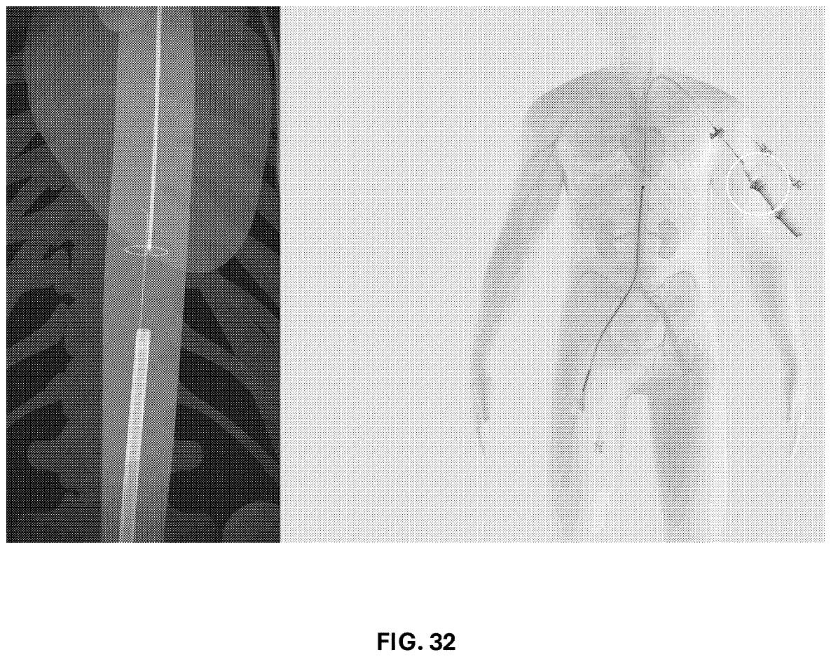

Advancing a guidewire through the first introducer sheath such that a end portion thereof exits the first introducer sheath at an aortic implantation site or proximate to the aortic implantation site and capturing the guidewire end portion with the medical loop snare, as illustrated in FIG. 32.

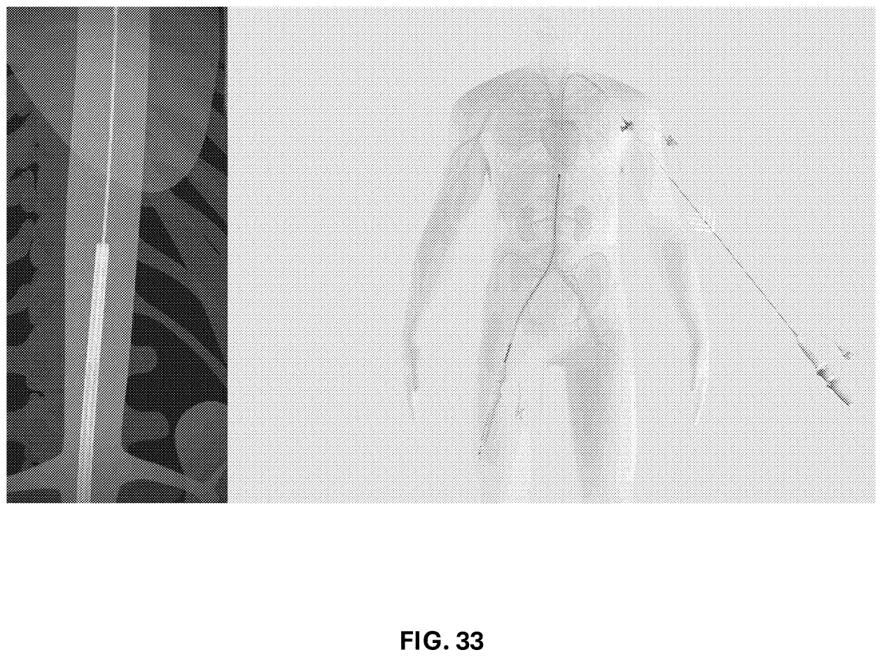

Withdrawing the medical loop snare through the second introducer sheath while maintaining the guidewire within the medical loop snare to advance the device body, including the pump unit and the drive unit, toward the implantation site, as sequentially illustrated in FIGS. 33-35.

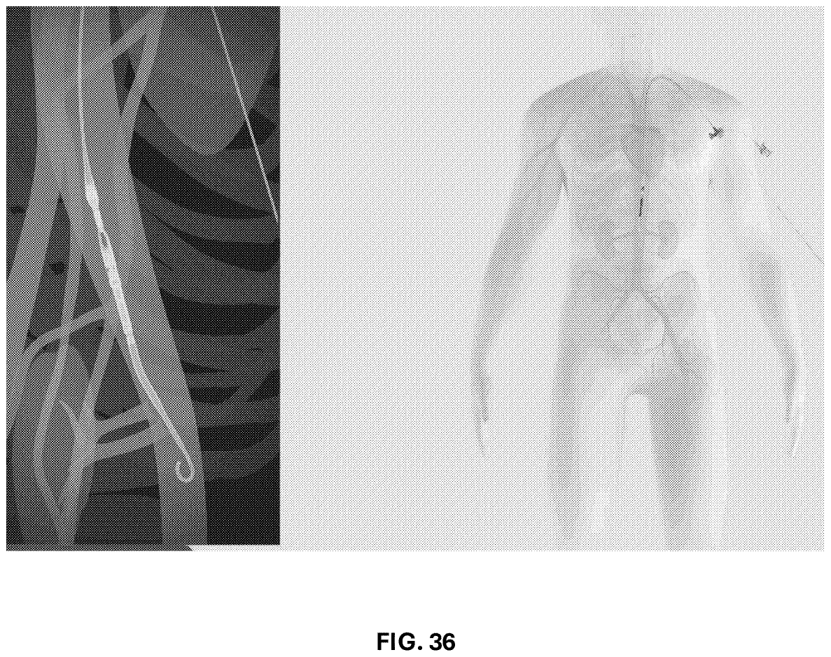

Removing the first introducer sheath from the femoral access site and closing the femoral access site, as illustrated in FIG. 36.

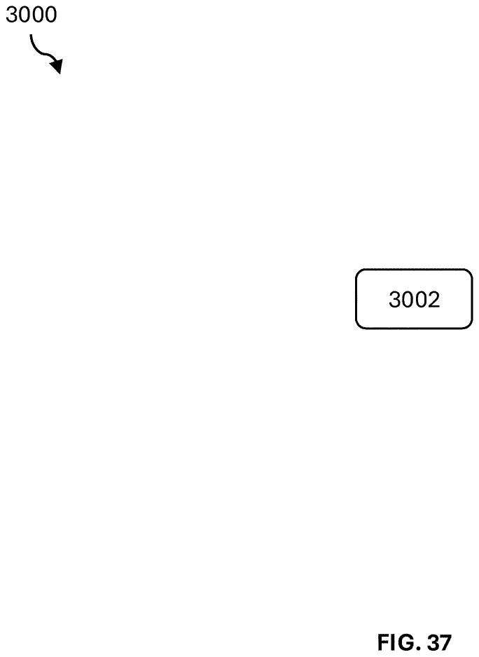

More particularly, referring to FIG. 37, the method 3000 includes advancing a medical device through a first access site of a subject's body and into a subject's lumen of the subject's body, at 3002, such that a first device portion of the medical device is externalized at least partially through a second access site of the subject's body, and a second device portion of the medical device is positioned at an implantation site within the subject's lumen. The first device portion may be externalized for example by pushing on the medical device through the first access site or by pulling on it through the second access site.

For example, the driveline 106 is introduced into a femoral access site of the subject's body, followed by the device body 118, including the pump unit 102 and the drive unit. Then, the driveline 106 is advanced along a subject's cardiovascular system up to an axillary or subclavian access site of the subject's body, where the driveline 106 is externalized, and the device body 118 is positioned at a vascular implantation site within the aorta lumen. The driveline 106 may be externalized for example by pushing on the device body 118 through the femoral access site or by pulling on it through the axillary or subclavian access site. In particular, the driveline 106 is advanced along the following vascular path: femoral artery, external iliac artery, common iliac artery, abdominal aorta, descending aorta, aortic arch, subclavian artery, and optionally the axillary artery.

According to the method 3000, a driveline, for example the driveline 106, as small as 4F in size may be externalized through the axillary or subclavian access site, which advantageously enables hemostasis by simple finger pressure of the axillary or subclavian access site and advantageously enables ambulatory subjects, compared to a driveline externalized through a femoral access site.

According to the method 3000, a driveline, for example the driveline 106, having a size of about 4F or smaller may be externalized through an axillary or subclavian access site. Such a configuration advantageously enables hemostasis by simple finger pressure at the access site and facilitates subject ambulation, compared to a driveline externalized through a femoral access site.

The vascular implantation site may include an implantation site in the aortic root, the ascending aorta, the aortic arch, and the thoracic (descending) aorta, including at proximal to the renal arteries (also referred herein to as a “suprarenal implantation site” or a “infrarenal position implantation site”).

The first access site may be located at an inferior anatomical location of the subject's body, and the second access site may be located at a superior anatomical location of the subject's body.

The first access site may be located at an inferior or superior anatomical location of the subject's body, and the second access site may be located on a subject's limb, for example on a subject's arm (also referred to herein as a “radial access”) or on a subject's leg.

The method 3000 optionally includes (i) introducing a first sheath into the first access site, and (ii) advancing the first device portion and the second device portion of the medical device through the first sheath.

For example, a first introducer sheath, for example one of a 14-18 French size, is introduced into the femoral access site, and the transcatheter implantable blood pump 100 is advanced through the first introducer sheath.

The method 3000 optionally includes advancing the first sheath within the subject's lumen to position a distal end portion of the first sheath at the implantation site or proximate to the implantation site.

For example, the first introducer sheath is advanced along the subject's cardiovascular system to position a distal end portion of the first introducer sheath at the vascular implantation site or proximate to the vascular implantation site.

The method 3000 optionally includes (i) introducing a second sheath into the second access site, and (ii) externalizing at least a portion of the first device portion from the subject's body through the second sheath.

For example, a second introducer sheath, for example one of a 3-5 French size, is introduced into the axillary or subclavian access site, and the driveline 106 is externalized through the axillary or subclavian access site through the second introducer sheath.

The method 3000 optionally includes advancing the second sheath within the subject's lumen to position a distal end portion of the second sheath at the implantation site or proximate to the implantation site. Such positioning of the distal end portion of the second sheath advantageously stabilize and/or immobilize the second portion of the medical device within the subject's lumen and advantageously prevent or mitigate vibration thereof.

For example, the second introducer sheath is advanced along the subject's cardiovascular system to position a distal end portion of the second introducer sheath at the vascular implantation site or proximate to the vascular implantation site. Such positioning of the distal end portion of the second sheath advantageously provides structural rigidity or stiffness to the driveline 106, preventing or reducing migration of the device body 118 due to buckling of the driveline 106.

The method 3000 optionally includes (i) advancing a retrieval instrument through the second sheath, which is inserted into the second access site, and toward the implantation site; (ii) capturing the first device portion with the retrieval instrument within the subject's lumen; and (iii) withdrawing the retrieval instrument from the second sheath to at least partially externalize the first device portion from the subject's body through the second sheath. Withdrawing the retrieval instrument from the second sheath optionally moves the second portion of the medical device within the first sheath and optionally position the second portion of the medical device at the implantation site or proximate to the implantation site.

For example, a medical loop snare is advanced toward the vascular implantation site through the second introducer sheath inserted into the axillary or subclavian access site. The medical loop snare is manipulated outside of the subject's body to capture the second capturable element 216, for example the second hook element 218, of the guidewire 208 within the subject's vascular system, for example located in the aortic arch. The medical loop snare is withdrawn through the second introducer sheath, thereby externalizing the driveline 106 from the axillary or subclavian access site through the second introducer sheath. These procedure may also be performed with the electrical driveline 206 or the driveline 106 free of the guidewire 208.

According to the method 3000, the second portion of the medical device is advanced within the first sheath, and eventually implanted in the subject's body, by withdrawing the retrieval instrument capturing the first second portion of the medical device from the first sheath. Such procedure advantageously reduces the transverse dimension of the first sheath since no overlapping sheath containing the medical device is required to be inserted within the first sheath for delivery or implantation purposes.

The method 3000 optionally includes holding the first device portion externalized through the second access site while removing the first sheath from the first access site to permit the second device portion to exit the first sheath within the subject's lumen, for example at the implantation site or proximate to the implantation site.

For example, the driveline 106 externalized through the axillary or subclavian access is held in place relative to the axillary or subclavian access site, while the first introducer sheath is removed from the femoral access site, permitting the device body 118 to exit the first introducer sheath within the subject's vascular system, for example at the vascular implantation site or proximate to the vascular implantation site.

The method 3000 optionally includes operatively coupling the first device portion, which is externalized at least partially through the second access site, to a console to operate the second device portion within the subject's lumen.

The method 3000 optionally includes transmitting operating power, for example electric (e.g., via an electric cable) or mechanical (e.g., via a driveshaft) power, through the first device portion, which is externalized at least partially through the second access site, to operate the second device portion within the subject's lumen.

The method 3000 optionally includes operating the second device portion during positioning of the second device portion at the implantation site.

For example, the electrical connector 210 of the driveline 106, which is externalized from the axillary or subclavian access site, is electrically connected to the console to operate the device body 118 within the subject's vasculature system, for example at the vascular implantation site. The device body 118 mat be operated during the implantation process to avoid device body-induced thrombogenesis.

The method 3000 optionally includes percutaneously establishing at least one of the first access site and the second access site, for example the femoral and/or axillary or subclavian access site(s).

The method 3000 optionally includes percutaneously establishing at least one of the first access site and the second access site, for example the femoral and/or axillary or subclavian access site(s).

According to the method 3000, the first device portion includes an elongated portion and a guidewire coupled to the elongated portion. The method 3000 optionally includes removing the guidewire from the elongated portion after at least a portion of the first device portion is externalized from the subject's body through the second access site.

For example, externalized from the axillary or subclavian access site, the guidewire 208 may be removed from the driveline 106, for example by cutting or disconnecting.

The method 3000 optionally includes removing the first sheath from the first access site.

For example, the first introducer sheath is removed from the femoral access site.

The method 3000 optionally includes closing the first access site.

For example, the femoral access site is close.

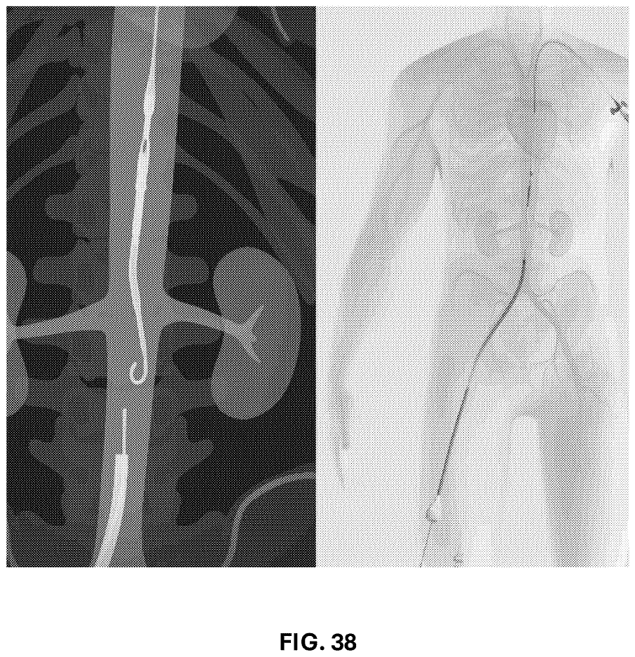

According to another aspect, as schematically represented in FIG. 38-42, this disclosure relates to a method 3100 of explanting a medical device from a subject's body, according to at least one embodiment. It is noted that reference to the transcatheter implantable blood pump 100, or any other feature described herein, is made for the sole purposes of describing how the explantation method 3100 of explanting may be implemented or practiced. Accordingly, such reference is not intended to limit the scope of the method 3100.

FIGS. 38-41 illustrate a sequential overview of an example of how the method 3100 may be performed to explant a medical device from a human aorta. The method 3100 may include the following steps.

Performing a femoral access to a subject's body, installing a first introducer sheath at the femoral access site, and advancing the first introducer sheath toward the medical device or proximate to the medical device. A second introducer sheath may already be installed at an axillary or subclavian access site of the subject's body and advanced to the medical device or proximate to the medical device.

Advancing a medical loop snare through the first introducer sheath toward the medical device implanted at an aortic implantation site or proximate to the medical device, as illustrated in FIG. 38.

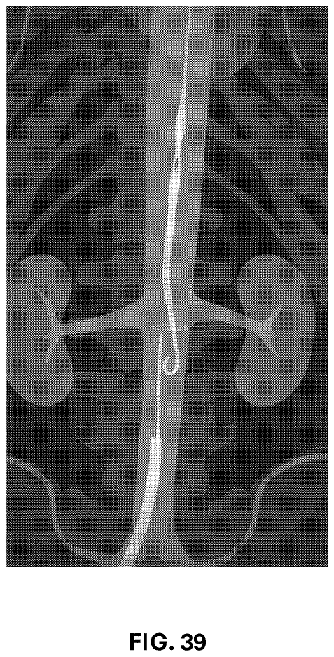

Capturing the medical device with the medical loop snare, as illustrated in FIG. 39.

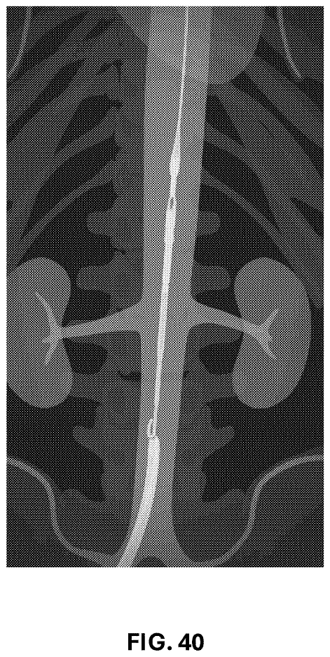

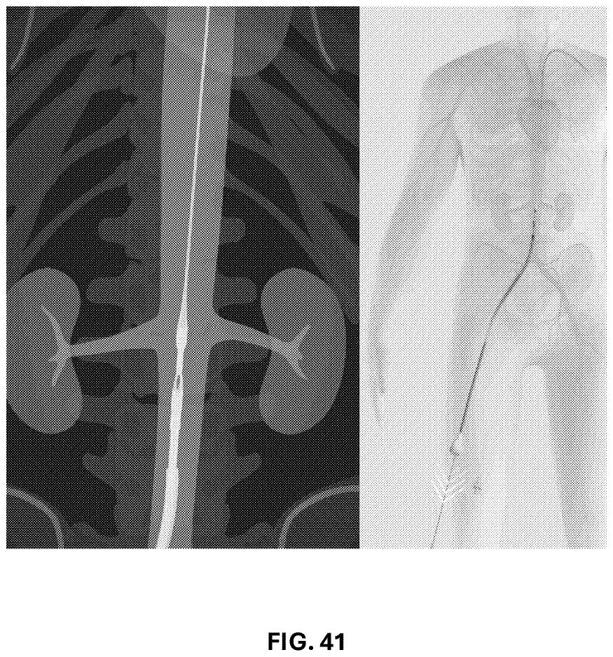

Withdrawing the medical loop snare through the first introducer sheath while maintaining the medical device within the medical loop snare so as to draw the medical device into the first introducer sheath, as sequentially illustrated in FIGS. 40-41.

Retrieving the medical device and removing the first introducer sheath from the femoral access site and the second introducer sheath from the axillary or subclavian access site.

Closing the femoral and the axillar or subclavian access site.

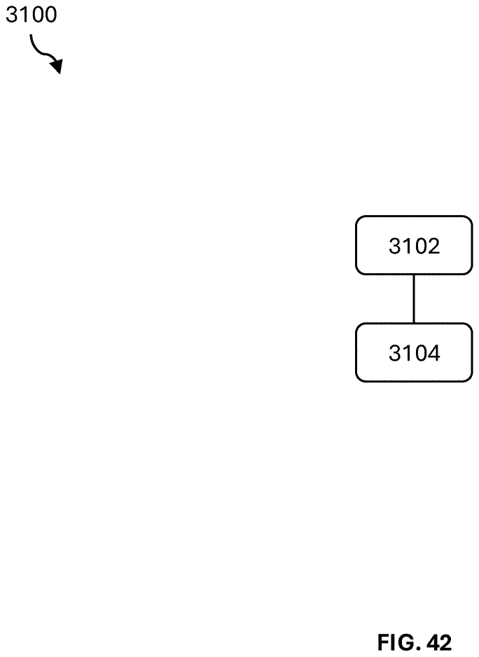

More particularly, referring to FIG. 42, according to the method 3100, a medical device includes a first device portion implanted in a subject's lumen of the subject's body, and a second device portion extending from the first device portion within the subject's lumen and externalized through a second access site of the subject's body. The method 3100 includes (i) establishing a first access site in communication with the subject's lumen, at 3102, and (ii) retrieving the first device portion from the subject's lumen through the first access site, at 3104, such that the second device portion is internalized through the second access site and retrieved through the first access site.

For example, according to the method 3100, the device body 118 is implanted in a subject's cardiovascular system, for example at an implantation site as described for the method 3000, and the driveline 106 extends from the device body 118 within the subject's cardiovascular system and is externalized through an axillary or subclavian access site of the subject's body.

The method 3100 includes (i) establishing a femoral access site in communication with the subject's cardiovascular system, and (ii) retrieving the device body 118 from the subject's cardiovascular system through the femoral access site, such that the driveline 106 is internalized through the axillary or subclavian access site and retrieved through the femoral access site.

According to the method 3100, the internalization and retrieval vascular path of the driveline 106 is the reverse of the vascular path described for the methos XIX. The implantation site as well as the first and second access sites are the same as described for the method 3000.

The method 3100 optionally includes percutaneously establishing first access site.

For example, the femoral access site is established. The femoral access site may be established at a same anatomical location as for the method 3000, for example on a same subject's leg, or at a different anatomical location than for the method 3000, for example the other subject's leg.

The method 3100 optionally includes introducing a first sheath into the first access site.

For example, a first introducer sheath, for example one of a 14-18 French size, is introduced into the femoral access site.

The method 3100 optionally includes advancing the first sheath along the subject's lumen to position a distal end portion of the first sheath at the implantation site or proximate to the implantation site.

For example, the first introducer sheath is advanced along the subject's cardiovascular system to position a distal end portion of the first introducer sheath at the vascular implantation site or proximate to the vascular implantation site.

The method 3100 optionally includes (i) advancing a retrieval instrument through the first sheath, which is inserted into the first access site, and toward the implantation site; (ii) capturing the second device portion with the retrieval instrument within the subject's lumen; and (iii) withdrawing the retrieval instrument from the first sheath to retrieve the second device portion through the first access site, internalize the first device portion through the second access site, and retrieve the first device portion through the first access site. According to the method 3100, the retrieval instrument captures a capturable element of the second device portion. Withdrawing the retrieval instrument from the first sheath causes the capturable element to transition from a capture configuration to a collapsed configuration.

For example, a medical loop snare is advanced toward the vascular implantation site through the first introducer sheath inserted into the femoral access site. The medical loop snare is manipulated outside of the subject's body to capture the first capturable element 920, for example the second hook element 218 of the driveline 106 within the subject's vascular system. The medical loop snare is withdrawn through the first introducer sheath, thereby retrieving the device body 118 and the driveline 106 from the femoral access site through the first introducer sheath. Withdrawn the medical loop snare through the first introducer sheath optionally causes the second capturable element 216, when provided as the second hook element 218, for example, to transition from a capture configuration to a collapsed configuration.

The method 3100 optionally includes operatively decoupling the first device portion, which is externalized at least partially through the second access site, from a console enabling operation of the second device portion.

For example, the electrical connector 210 of the driveline 106 is electrically disconnected from a console enabling operation of the device body 118.

The method 3100 optionally includes stopping a transmission of operating power through the first device portion, which is externalized at least partially through the second access site, wherein the operating power operates the second device portion.

For example, electrical transmission through the electrical driveline 206, which is externalized through the axillary or subclavian access site and operates the device body 118, is stopped.

The method 3100 optionally includes continuing operating the second device portion while the second device portion is being retrieved from the subject's lumen.

For example, the device body 118 is operated during the explantation or retrieval procedure.

The method 3100 optionally includes removing 1^{st} sheath from 1^{st} access site.

For example, the first introducer sheath is removed from the femoral access site.

The method 3100 optionally includes closing the first access site and the second access site.

For example, the femoral access site is closed.

### ADDITIONAL CLAUSES

### First Clause Set

1. A transcatheter implantable blood pump, comprising:
a pump unit;
a drive unit configured to drive the pump unit, the drive unit defining a longitudinal pump unit axis; and
a driveline configured to transmit operating power to the drive unit to drive the pump unit,
wherein the driveline is associated with the pump unit.
   2. The transcatheter implantable blood pump according to clause 1, wherein the driveline extends along the pump unit and is coupled thereto.
   3. The transcatheter implantable blood pump according to any one of clauses 1-2, wherein the pump unit defines a pump unit guide configured to receive the driveline.
   4. The transcatheter implantable blood pump according to any one of clauses 1-3, wherein the driveline is maintained in physical contact with the pump unit.
   5. The transcatheter implantable blood pump according to any one of clauses 1-4, wherein the driveline is secured to the pump unit.
   6. The transcatheter implantable blood pump according to any one of clauses 1-5, wherein the pump unit comprises a pump unit inlet wall defining a pump unit inlet, the driveline extending along the pump unit inlet wall between a first inlet wall end portion and a second inlet wall end portion of the pump unit inlet wall and being connected thereto.
   7. The transcatheter implantable blood pump according to clause 6, wherein the pump unit inlet wall defines an inlet guide configured to receive the driveline.
   8. The transcatheter implantable blood pump according to any one of clauses 6-7, wherein the driveline is maintained physical contact with the pump unit inlet wall.
   9. The transcatheter implantable blood pump according to any one of clauses 6-8, wherein the driveline is secured to the pump unit inlet wall.
   10. The transcatheter implantable blood pump according to any one of clauses 6-9, wherein the pump unit comprises a plurality of inlet arm members defining a plurality of pump unit sub-inlets therebetween, the pump unit sub-inlets being in fluid communication with the pump unit inlet, the driveline extending along at least one inlet arm member of the plurality of inlet arm members and being connected thereto.
   11. The transcatheter implantable blood pump according to clause 10, wherein at least one inlet arm member of the plurality of inlet arm members defines an inlet arm guide configured to receive the driveline.
   12. The transcatheter implantable blood pump according to any one of clauses 10-11, wherein the driveline is maintained physical contact with at least one inlet arm member of the plurality of inlet arm members.
   13. The transcatheter implantable blood pump according to any one of clauses 10-12, wherein the driveline is secured to at least one inlet arm member of the plurality of inlet arm members.
   14. The transcatheter implantable blood pump according to any one of clauses 10-13, wherein the driveline is fixedly attached to at least one inlet arm member of the plurality of inlet arm members.
   15. The transcatheter implantable blood pump according to any one of clauses 10-14, wherein the plurality of inlet arm members converges at a junction point disposed upstream of the pump unit inlet, and the driveline extends through the junction point.
   16. The transcatheter implantable blood pump according to any one of clauses 6-15, wherein the pump unit inlet has an opening substantially centered about the longitudinal pump unit axis and configured to direct blood axially relative to the longitudinal pump unit axis into a pump unit inner passage.
   17. The transcatheter implantable blood pump according to any one of clauses 6-16, wherein the driveline extends away from the pump unit inlet wall.
   18. The transcatheter implantable blood pump according to any one of clauses 1-17, wherein the pump unit comprises a pump unit outlet wall defining a pump unit outlet, the driveline extending along the pump unit outlet wall between a first outlet wall end portion and a second outlet wall end portion of the pump unit outlet wall and being connected thereto.
   19. The transcatheter implantable blood pump according to clause 18, wherein the pump unit outlet wall defines an outlet guide configured to receive the driveline.
   20. The transcatheter implantable blood pump according to any one of clauses 18-19, wherein the driveline is maintained physical contact with the pump unit outlet wall.
   21. The transcatheter implantable blood pump according to any one of clauses 18-20, wherein the driveline is secured to the pump unit outlet wall.
   22. The transcatheter implantable blood pump according to any one of clauses 18-21, wherein the pump unit comprises a plurality of outlet arm members defining a plurality of pump unit sub-outlets therebetween, the pump unit sub-outlets being in fluid communication with the pump unit outlet, the driveline extending along at least one outlet arm member of the plurality of outlet arm members and being connected thereto.
   23. The transcatheter implantable blood pump according to clause 22, wherein at least one outlet arm member of the plurality of outlet arm members defines an outlet arm guide configured to receive the driveline.
   24. The transcatheter implantable blood pump according to any one of clauses 22-23, wherein the driveline is maintained in physical contact with at least one outlet arm member of the plurality of outlet arm members.
   25. The transcatheter implantable blood pump according to any one of clauses 22-24, wherein the driveline is secured to at least one outlet arm member of the plurality of outlet arm members.
   26. The transcatheter implantable blood pump according to any one of clauses 22-25, wherein the driveline is fixedly attached to at least one outlet arm member of the plurality of outlet arm members.
   27. The transcatheter implantable blood pump according to any one of clauses 18-26, wherein the impeller comprises a downstream taper surface disposed at an axially downstream end of the impeller and oriented obliquely relative to the longitudinal pump unit axis, the downstream taper surface being configured to redirect at least a portion of blood flow radially outward relative to the longitudinal pump unit axis through the pump unit outlet.
   28. The transcatheter implantable blood pump according to any one of clauses 1-27, wherein the pump unit includes a first pump unit end portion and a second pump unit end portion, and the drive unit includes a first drive unit end portion and a second drive unit end portion; the second pump unit end portion being coupled to the first drive unit end portion; the pump unit inlet being disposed at the first pump unit end portion, and the pump unit outlet being disposed at the second pump unit end portion.
   29. The transcatheter implantable blood pump according to any one of clauses 18-28, wherein the pump unit comprises a shroud and an impeller; the shroud including a shroud wall corresponding to a pump unit wall defining the pump unit inlet, the pump unit outlet, and a pump unit passage therebetween; the impeller being disposed within the pump unit passage between the pump unit inlet and the pump unit outlet.
   30. The transcatheter implantable blood pump according to clause 29, wherein the drive unit comprises an electric motor 204 configured to rotate the impeller.
   31. The transcatheter implantable blood pump according to any one of clauses 29-30, wherein the driveline is an electrical driveline.

### Second Clause Set

1. A transcatheter implantable medical device, comprising:
a device body having a first body end portion and a second body end portion, and defining a device body longitudinal axis; and
a first contact element extending from the second body end portion, the first contact element including at least two atraumatic contact portions configured to atraumatically contact a subject's lumen wall of a subject's lumen of a subject's body.
   2. The transcatheter implantable medical device according to clause 1, wherein the first contact element is configured to transition between a delivery configuration for advancing the transcatheter implantable medical device within the subject's lumen, and a contact configuration in which the first contact element is configured to atraumatically contact the subject's lumen wall.
   3. The transcatheter implantable medical device according to clause 2, wherein, in the delivery configuration, the first contact element extends substantially parallel to the device body longitudinal axis.
   4. The transcatheter implantable medical device according to any one of clauses 2-3, wherein, in the contact configuration, the first contact element extends transverse to the device body longitudinal axis.
   5. The transcatheter implantable medical device according to any one of clauses 2-4, wherein the first contact element is biased toward the contact configuration from the delivery configuration.
   6. The transcatheter implantable medical device according to any one of clauses 2-5, wherein the first contact element comprises a terminal portion and a deflection portion extending between the second body end portion and the terminal portion, the deflection portion being configured to transition the terminal portion between a first terminal portion configuration in which the terminal portion is disposed at a location axially distal relative to the drive along the device body longitudinal axis, and a second terminal portion configuration in which the terminal portion is disposed at a location radially outward relative to the device body longitudinal axis.
   7. The transcatheter implantable medical device according to clause 6, wherein the terminal portion is in the first terminal portion configuration when the first contact element is in the delivery configuration.
   8. The transcatheter implantable medical device according to any one of clauses 6-7, wherein the terminal portion is in the second terminal portion configuration when the first contact element is in the contact configuration.
   9. The transcatheter implantable medical device according to any one of clauses 6-8, wherein the terminal portion comprises a first atraumatic contact portion of the at least two atraumatic contact portions.
   10. The transcatheter implantable medical device according to any one of clauses 6-9, wherein the deflection portion comprises a second atraumatic contact portion of the at least two atraumatic contact portions.
   11. The transcatheter implantable medical device according to any one of clauses 6-10, wherein the first contact element includes exactly two atraumatic contact portions, the deflection portion comprising a first atraumatic contact portion of the two atraumatic contact portions, and the terminal portion comprising a second atraumatic contact portion of the two atraumatic contact portions.
   12. The transcatheter pump according to any one of claims 6-11, wherein the deflection portion includes a segment extending substantially coaxially from the second body end portion along the device body longitudinal axis.
   13. The transcatheter implantable medical device according to any one of clauses 6-12, wherein the terminal portion comprises a capturable element configured to be captured by a retrieval instrument within the subject's lumen.
   14. The transcatheter implantable medical device according to clause 13, wherein the capturable element is configured to transition between a delivery configuration for advancing the transcatheter implantable medical device within the subject's lumen, and a capture configuration in which the capturable element is configured to be captured by the retrieval instrument within the subject's lumen.
   15. The transcatheter implantable medical device according to clause 14, wherein, in the delivery configuration, the capturable element extends substantially parallel to the device body longitudinal axis.
   16. The transcatheter implantable medical device according to any one of clauses 14-15, wherein the capturable element is biased toward the capture configuration.
   17. The transcatheter implantable medical device according to any one of clauses 14-16, wherein the capturable element is configured to transition between the capture configuration and a captured configuration in which the capturable element has a reduced radial profile relative to the capture configuration.
   18. The transcatheter implantable medical device according to clause 17, wherein, in the captured configuration, the capturable element is collapsed or folded upon itself.
   19. The transcatheter implantable medical device according to any one of clauses 14-18, wherein, when the capturable element is in the delivery configuration, the first contact element is in the delivery configuration.
   20. The transcatheter implantable medical device according to any one of clauses 14-19, wherein, when the capturable element is in the capture configuration, the first contact element is in the contact configuration.
   21. The transcatheter implantable medical device according to any one of clauses 14-20, wherein the capturable element can be transitioned between the delivery configuration and the capture configuration, and the first contact element can be transitioned between the delivery configuration and the contact configuration independently from one another.
   22. The transcatheter implantable medical device according to any one of clauses 14-21, wherein, when the first contact element is in the contact configuration and the capturable element is in the capture configuration, the first contact element is configured to position the capturable element spaced apart from the subject's lumen wall.
   23. The transcatheter implantable medical device according to clause 22, wherein the terminal portion comprise a transition segment configured to position the capturable element spaced apart from the subject's lumen wall.
   24. The transcatheter implantable medical device according to any one of clauses 22-23, wherein the capturable element comprises a hook element including an end tip portion, the hook element defining a reference plane, the end tip portion being positioned outside the reference plane.
   25. The transcatheter implantable medical device according to any one of clauses 1-24, further comprising a second contact element extending from the first body end portion, the second contact element including at least one atraumatic contact portion configured to atraumatically contact the subject's lumen wall of the subject's lumen of a subject's body.
   26. The transcatheter implantable medical device according to clause 25, wherein the second contact element comprises a contact portion and a positioning portion extending between the first body end portion and the contact portion, the positioning portion being configured to position and maintain the contact portion away from the first body end portion and substantially coaxial with the device body longitudinal axis.
   27. The transcatheter implantable medical device according to clause 26, wherein the at least one atraumatic contact portion is provided on the contact portion of the second contact element.
   28. The transcatheter pump according to any one of claims 26-27, wherein the positioning portion includes a segment extending substantially coaxially from the first shroud end portion along the longitudinal shroud axis.
   29. The transcatheter implantable medical device according to any one of clauses 25-28, wherein the first contact element in the contact configuration and the second contact element are configured such that the device body longitudinal axis is oriented obliquely relative to a longitudinal axis defined by the subject's lumen.
   30. The transcatheter implantable medical device according to any one of clauses 26-29, further comprising a driveline configured to extend away from the first body end portion.
   31. The transcatheter implantable medical device according to clause 30, wherein the driveline is associated with the second contact element.
   32. The transcatheter implantable medical device according to any one of clauses 30-31, wherein the driveline extends at least partially along the positioning portion and the contact portion and is coupled thereto.
   33. The transcatheter implantable medical device according to any one of clauses 30-32, wherein the driveline is configured to enable operation of the device body.
   34. The transcatheter implantable medical device according to any one of clauses 30-33, wherein the driveline comprises a first driveline portion and a second driveline portion operatively connectable to the first driveline portion at an operative connection, the contact portion being configured to receive the operative connection.
   35. The transcatheter implantable medical device according to clause 34, wherein the contact portion has a profiled body having an apex portion oriented away from the positioning portion, the first driveline portion extending from the apex portion.

### Third Clause Set

1. A transcatheter implantable blood pump, comprising:
an impeller defining an impeller internal passage extending at least partially between a first impeller end portion and a second impeller end portion thereof;
a drive configured to rotate the impeller; and
a first hydrodynamic bearing including an impeller vaned bearing member and a drive bearing member; the impeller vaned bearing member including a base surface and a plurality of vanes extending therealong; the plurality of vanes defining a plurality of vane passages in fluid communication with a port of the impeller internal passage opening onto the base surface,
wherein rotation of the impeller generates blood flow through the impeller internal passage and through the plurality of vane passages to generate hydrodynamic support between the impeller vaned bearing member and the drive bearing member.
   2. The transcatheter implantable blood pump according to clause 1, wherein at least one vane of the plurality of vanes extending radially outward on the base surface from the port to an outer periphery of the impeller vaned bearing member along an entire radial length therebetween.
   3. The transcatheter implantable blood pump according to any one of clauses 1-2, wherein the plurality of vanes of the impeller vaned bearing member collectively defines a segmented bearing surface.
   4. The transcatheter implantable blood pump according to cause 3, wherein the drive bearing member defines an unsegmented bearing surface.
   5. The transcatheter implantable blood pump according to clause 4, wherein a central impeller region of the impeller vaned bearing member includes a first sub-surface, and a central drive region of the drive bearing member includes a second sub-surface.
   6. The transcatheter implantable blood pump according to clause 5, wherein the first bearing surface is concave, and the second bearing surface is convex.
   7. The transcatheter implantable blood pump according to clause 5, wherein the first bearing surface is convex, and the second bearing surface is concave.
   8. The transcatheter implantable blood pump according to clause 5, wherein the first bearing surface is flat, and the second bearing surface is flat.
   9. The transcatheter implantable blood pump according to any one of clauses 5-8, wherein a peripheral impeller region of the impeller vaned bearing member includes a third sub-surface, and a peripheral drive region of the drive bearing member includes a fourth sub-surface.
   10. The transcatheter implantable blood pump according to clause 9, wherein the third bearing surface is concave, and the fourth bearing surface is convex.
   11. The transcatheter implantable blood pump according to clause 9, wherein the third bearing surface is convex, and the fourth bearing surface is concave.
   12. The transcatheter implantable blood pump according to clause 9, wherein the third bearing surface is flat, and the fourth bearing surface is flat.
   13. The transcatheter implantable blood pump according to any one of clauses 5-8, wherein the plurality of vanes includes a plurality of first vane surfaces collectively defining the first sub-surface; at least one first vane surface of the plurality of first vane surfaces including a surface edge facing the port, and a spacing wall extending from the bearing surface to the surface edge.
   14. The transcatheter implantable blood pump according to clause 1-13, wherein at least one of the first sub-surface and the third sub-surface of at least one vane of the plurality of vanes defines an impeller rotation leading wedge surface.
   15. The transcatheter implantable blood pump according to clause 1-14, wherein at least one of the first sub-surface and the third sub-surface of at least one vane of the plurality of vanes defines an impeller rotation tailing wedge surface.
   16. The transcatheter implantable blood pump according to any one of clauses 1-15, wherein another port of the impeller internal passage opens onto the impeller hub, between the first impeller end portion and the second impeller end portion thereof.
   17. The transcatheter implantable blood pump according to any one of clauses 1-16, wherein the impeller and the plurality of vanes are configured to draw blood in a same axial direction.
   18. The transcatheter implantable blood pump according to clause 18, wherein a magnetic attractive force between the first magnetic element and the second magnetic element opposes a thrust force of the impeller.
   19. The transcatheter implantable blood pump according to any one of clauses 1-18, further comprising:
a shroud configured to be coupled to the drive and to at least partially enclose the impeller; and
a second hydrodynamic bearing including an impeller bearing member disposed at the first impeller end portion and having a cylindrical body and, and a shroud bearing member disposed at the shroud and having an obliquely truncated tubular body configured to rotatably receive the cylindrical body therein.
   20. The transcatheter implantable blood pump according to clause 19, wherein rotation of the impeller causes an outer surface of the cylindrical body to be cyclically exposed to an inner surface of the obliquely truncated tubular body and an environment outside of the interface between the inner surface and the outer surface.

### Fourth Clause Set

1. A method of implanting a medical device within a subject's body, the method comprising:
advancing the medical device through a first access site of the subject's body and into a subject's lumen of the subject's body,
wherein a first device portion of the medical device is externalized at least partially through a second access site of the subject's body, and a second device portion of the medical device is positioned at an implantation site within the subject's lumen.
   2. The method according to clause 1, further comprising introducing a first sheath into the first access site and advancing the first device portion and the second device portion of the medical device through the first sheath.
   3. The method according to clause 2, further comprising advancing the first sheath within the subject's lumen to position a distal end portion of the first sheath at the implantation site or proximate to the implantation site.
   4. The method according to any one of clauses 1-3, further comprising:
introducing a second sheath into the second access site, and
externalizing at least a portion of the first device portion from the subject's body through the second sheath.
   5. The method according to clause 4, further comprising advancing the second sheath within the subject's lumen to position a distal end portion of the second sheath at the implantation site or proximate to the implantation site.
   6. The method according to clause 5, wherein the implantation site is an aortic segment selected from the group consisting of an aortic root, an ascending aorta, an aortic arch, and a descending aorta.
   7. The method according to clause 6, wherein the implantation site is within the descending aorta proximal to the renal arteries.
   8. The method according to any one of clauses 4-7, further comprising:
advancing a retrieval instrument through the second sheath, which is inserted into the second access site, and toward the implantation site;
capturing the first device portion with the retrieval instrument within the subject's lumen; and
withdrawing the retrieval instrument from the second sheath to at least partially externalize the first device portion from the subject's body through the second sheath.
   9. The method according to clause 7, wherein said withdrawing the retrieval instrument from the second sheath moves the second portion of the medical device within the first sheath.
   10. The method according to any one of clauses 7-8, wherein said withdrawing the retrieval instrument from the second sheath positions the second portion of the medical device at the implantation site or proximate to the implantation site.
   11. The method according to any one of clauses 1-9, further comprising operatively coupling the first device portion, which is externalized at least partially through the second access site, to a console to operate the second device portion within the subject's lumen.
   12. The method according to any one of clauses 1-10, further comprising transmitting operating power through the first device portion, which is externalized at least partially through the second access site, to operate the second device portion within the subject's lumen.
   13. The method according to clause 11, further comprising operating the second device portion during positioning of the second device portion at the implantation site.
   14. The method according to any one of clauses 1-12, further comprising percutaneously establishing at least one of the first access site and the second access site.
   15. The method according to any one of clauses 1-13, wherein the subject's lumen is a blood vessel lumen, and the first access site and the second access site communicate with the blood vessel lumen.
   16. The method according to clause 14, wherein one of the first access site and the second access site is located at an inferior anatomical location of the subject's body, and the other one of the first access site and the second access site is located at a superior anatomical location of the subject's body.
   17. The method according to clause 15, wherein the access site located at the inferior anatomical location communicates with a femoral artery lumen, and the access site located at the superior anatomical location communicates with an axillary artery lumen or a subclavian artery lumen.
   18. The method according to clause 14, wherein one of the first access site and the second access site is located at an inferior anatomical location of the subject's body, and the other one of the first access site and the second access site communicates with a radial artery lumen.
   19. The method according to clause 15, wherein one of the first access site and the second access site is located at a superior anatomical location of the subject's body, and the other one of the first access site and the second access site communicates with a radial artery lumen.
   20. The method according to any one of clauses 1-18, wherein the first device portion comprises an elongated portion and a guidewire coupled to the elongated portion, the method further comprising removing the guidewire from the elongated portion after at least a portion of the first device portion is externalized from the subject's body through the second access site.
   21. The method according to any one of clauses 2-19, further comprising removing the first sheath from the first access site.
   22. The method according to clause 20, further comprising holding the first device portion at the second access site while removing the first sheath from the first access site to permit the second device portion to exit the first sheath within the subject's lumen.
   23. The method according to any one of clauses 1-21, further comprising closing the first access site.
   24. The method according to any one of clauses 1-22, wherein the first device portion is a driveline configured to transmit operating power to operate the second device portion within the subject's lumen.
   25. The method according to clause 23, wherein the driveline is an electrical driveline configured to transmit electrical power to the second device portion to operate the second device portion within the subject's lumen.
   26. The method according to any one of clauses 23-24, wherein the second device portion is electrically operable.
   27. The method according to clause 25, wherein the second device portion comprises an electrically operable pump.
   28. The method according to clause 26, wherein the electrically operable pump comprises a pump unit comprising an impeller and a shroud at least partially enclosing the impeller; and a drive unit configured to receive electrical power from the electrical driveline to rotate the impeller, wherein at least a portion of the electrical driveline is associated with at least a portion of the shroud.
   29. The method according to clause 27, wherein the at least a portion of the electrical driveline extends along the at least a portion of the shroud.
   30. The method according to clause 28, wherein the shroud comprises a shroud inlet, the at least a portion of the electrical driveline extending along at least a portion of the shroud inlet.
   31. The method according to clause 29, wherein the shroud inlet faces axially toward an upstream direction of blood flow.

### Fifth Clause Set

1. A method of explanting a medical device from a subject's body, wherein the medical device comprises a first device portion implanted in a subject's lumen of the subject's body, and a second device portion extending from the first device portion within the subject's lumen and externalized through a second access site of the subject's body, the method comprising:
establishing a first access site in communication with the subject's lumen; and
retrieving the first device portion from the subject's lumen through the first access site,
wherein the second device portion is internalized through the second access site and retrieved through the first access site.
   2. The method according to clause 1, further comprising percutaneously establishing first access site.
   3. The method according to any one of clauses 1-2, further comprising introducing a first sheath into the first access site.
   4. The method according to clause 3, further comprising advancing the first sheath along the subject's lumen to position a distal end portion of the first sheath at the implantation site or proximate to the implantation site.
   5. The method according to any one of clauses 3-4, further comprising:
advancing a retrieval instrument through the first sheath, which is inserted into the first access site, and toward the implantation site;
capturing the second device portion with the retrieval instrument within the subject's lumen; and
withdrawing the retrieval instrument from the first sheath to retrieve the second device portion through the first access site, internalize the first device portion through the second access site, and retrieve the first device portion through the first access site.
   6. The method according to clause 5, wherein the retrieval instrument captures a capturable element of the second device portion, said withdrawing the retrieval instrument from the first sheath causes the capturable element to transition from a capture configuration to a collapsed configuration.
   7. The method according to any one of clauses 1-6, further comprising operatively decoupling the first device portion, which is externalized at least partially through the second access site, from a console enabling operation of the second device portion.
   8. The method according to any one of clauses 1-7, further comprising stopping a transmission of operating power through the first device portion, which is externalized at least partially through the second access site, wherein the operating power operates the second device portion.
   9. The method according to clause 8, further comprising continuing operating the second device portion while the second device portion is being retrieved from the subject's lumen.
   10. The method according to any one of clauses 3-9, further comprising removing the first sheath from the first access site.
   11. The method according to any one of clauses 1-10, further comprising closing the first access site and the second access site.
   12. The method according to any one of clauses 1-11, wherein the subject's lumen is a blood vessel lumen, and the first access site and the second access site communicates with the blood vessel lumen.
   13. The method according to clause 12, wherein one of the first access site and the second access site is located at an inferior anatomical location of the subject's body, and the other one of the first access site and the second access site is located at a superior anatomical location of the subject's body.
   14. The method according to clause 13, wherein the access site located at the inferior anatomical location communicates with a femoral artery lumen, and the access site located at the superior anatomical location communicates with an axillary artery or a subclavian artery lumen.
   15. The method according to clause 12, wherein one of the first access site and the second access site is located at an inferior anatomical location of the subject's body, and the other one of the first access site and the second access site communicates with a radial artery lumen.
   16. The method according to clause 15, wherein one of the first access site and the second access site is located at a superior anatomical location of the subject's body, and the other one of the first access site and the second access site communicates with a radial artery lumen.
   17. The method according to any one of clauses 1-1816 wherein the second medical portion is implanted within an aortic segment selected from the group consisting of an aortic root, an ascending aorta, an aortic arch, and a descending aorta.
   18. The method according to clause 17, wherein the second medical portion is implanted within the descending aorta at a position proximal to the renal arteries.
   19. The method according to any one of clauses 1-18, wherein the first device portion is a driveline configured to transmit operating power to the second device portion.
   20. The method according to clause 19, wherein the driveline is an electrical driveline configured to transmit electrical power to the second device portion.
   21. The method according to any one of clauses 19-20, wherein the second device portion is configured to be electrically operated.
   22. The method according to clause 21, wherein the second device portion comprises an electrically operable pump.
   23. The method according to clause 22, wherein the electrically operable pump comprises: a pump unit comprising an impeller and a shroud at least partially enclosing the impeller; and a drive unit configured to be electrically driven through the electrical driveline to rotate the impeller, wherein at least a portion of the electrical driveline is associated with at least a portion of the shroud.
   24. The method according to clause 23, wherein the at least a portion of the electrical driveline extends along the at least a portion of the pump unit.
   25. The method according to clause 24, wherein the shroud comprises a shroud inlet, the at least a portion of the electrical driveline extends along at least a portion of the shroud inlet.
   26. The method according to clause 25, wherein the shroud inlet faces axially toward an upstream direction of blood flow.

1. A transcatheter blood pump implantable within a subject's lumen defined by a subject's lumen wall, the transcatheter blood pump comprising:
an impeller having an axis of rotation;
a shroud having a first shroud end portion, a second shroud end portion, and a shroud internal passage extending between the first shroud end portion and the second shroud end portion, the impeller disposed within the shroud internal passage;
an electric motor configured to rotate the impeller, the electric motor having a first motor end portion and a second motor end portion, the first motor end portion coupled to the second shroud end portion; and
an electric driveline configured to transmit operating power to the electric motor to rotate the impeller,
wherein the electric driveline extends along at least one of the shroud and the electric motor and is coupled thereto.

2. The transcatheter blood pump according to claim 1, wherein the shroud comprises a shroud inlet disposed at the first shroud end portion and a shroud outlet disposed at the second shroud end portion, the electric driveline extending along a portion of the shroud defining the shroud outlet and connected thereto.

3. The transcatheter blood pump according to claim 2, wherein the shroud comprises a plurality of outlet arm members defining a plurality of shroud sub-outlets therebetween, the plurality of sub-outlets associated with the shroud outlet, the electric driveline extending along at least one outlet arm member of the plurality of outlet arm members and connected thereto.

4. The transcatheter blood pump according to claim 2, wherein the impeller comprises a tapered surface disposed at an axially downstream end of the impeller, the tapered surface configured to redirect at least a portion of blood flow radially outward relative to the axis of rotation of the impeller, through the shroud outlet.

5. The transcatheter blood pump according to claim 2, wherein the electric driveline extends along a portion of the shroud defining the shroud inlet and is connected thereto.

6. The transcatheter blood pump according to claim 2, wherein the shroud inlet defines an inlet opening substantially centered about the axis of rotation of the impeller and configured to intake blood axially relative to the impeller rotation axis, into the shroud internal passage.

7. The transcatheter blood pump according to claim 1, wherein the electric driveline is configured to connect to at least one of the first motor end portion and the second motor end portion to transmit power to the electric motor to rotate the impeller.

8. The transcatheter blood pump according to claim 1, wherein the electric driveline extends along the shroud.

9. The transcatheter blood pump according to claim 1, further comprising a first contact element extending from the second motor end portion, the first contact element including at least two atraumatic contact portions configured to atraumatically contact the subject's lumen wall within the subject's lumen.

10. The transcatheter blood pump according to claim 9, wherein the first contact element is configured to transition between a delivery configuration for advancing the transcatheter blood pump within the subject's lumen and a contact configuration in which the at least two atraumatic contact portions are configured to atraumatically contact the subject's lumen wall.

11. The transcatheter blood pump according to claim 10, wherein the first contact element comprises a terminal portion and a deflection portion extending between the second motor end portion and the terminal portion, the deflection portion configured to transition the terminal portion between a first terminal portion configuration in which the terminal portion is disposed at a location axially distal relative to the electric motor, along a longitudinal motor axis defined by the electric motor, and a second terminal portion configuration in which the terminal portion is disposed at a location radially outward relative to the longitudinal motor axis.

12. The transcatheter blood pump according to claim 11, wherein the deflection portion includes a segment extending substantially coaxially from the second motor end portion along the longitudinal motor axis.

13. The transcatheter blood pump according to claim 11, wherein the terminal portion comprises a capturable element configured to be captured by a retrieval instrument within the subject's lumen.

14. The transcatheter blood pump according to claim 13, wherein the capturable element is disposed at a distal-most end of the terminal portion.

15. The transcatheter blood pump according to claim 13, wherein the first contact element comprises a transition segment configured to maintain the capturable element spaced apart from the subject's lumen wall for capturing the capturable element within the subject's lumen.

16. The transcatheter blood pump according to claim 10, wherein the first contact element does not define any portion of the shroud internal passage.

17. The transcatheter blood pump according to claim 9, further comprising a second contact element extending from the first shroud end portion, the second contact element including at least one atraumatic contact portion configured to atraumatically contact the subject's lumen wall within the subject's lumen.

18. The transcatheter blood pump according to claim 17, wherein the second contact element comprises a contact portion and a positioning portion extending between the first shroud end portion and the contact portion, the positioning portion being configured to position the contact portion at a location axially distal relative to the shroud, along a longitudinal shroud axis defined by the shroud.

19. The transcatheter blood pump according to claim 18, wherein the positioning portion includes a segment extending substantially coaxially from the first shroud end portion, along the longitudinal shroud axis.

20. The transcatheter blood pump according to claim 18, wherein at least one of the positioning portion and the contact portion includes a portion of the electric driveline.

21. The transcatheter blood pump according to claim 18, wherein the portion of the electric driveline is a first electric driveline portion, the electric driveline comprising a second electric driveline portion, an operative electrical connection between the first electric driveline portion and the second electric driveline portion being disposed at the contact portion.

22. The transcatheter blood pump according to claim 17, wherein the first contact element and the second contact element are configured such that, when the transcatheter blood pump is implanted within a substantially straight segment of the subject's lumen, a common longitudinal axis defined by the shroud and the electric motor is oriented obliquely relative to a longitudinal axis defined by the substantially straight segment of the subject's lumen.

23. The transcatheter blood pump according to claim 1, wherein the impeller comprises an impeller hub inlet, an impeller hub outlet, and an impeller internal passage in fluid communication with the impeller hub inlet and the impeller hub outlet, the transcatheter blood pump further comprising:
a hydrodynamic bearing including a first bearing member and a second bearing member;
the first bearing member being associated with the impeller and defining a first bearing surface, the impeller hub outlet opening onto the first bearing surface, the first bearing member including a plurality of vanes defining a plurality of vane passages in fluid communication with the impeller internal passage; and
the second bearing member being associated with the electric motor and defining a second bearing surface,
wherein the impeller is configured to generate blood flow through the impeller internal passage and through the plurality of vane passages to generate hydrodynamic support between the first bearing member and the second bearing member.

24. The transcatheter blood pump according to claim 23, wherein at least one vane of the plurality of vanes extends from the impeller hub outlet toward an outer periphery of the first bearing surface.

25. The transcatheter blood pump according to claim 23, wherein the first bearing surface is segmented by the plurality of vane passages.

26. The transcatheter blood pump according to claim 24, wherein the second bearing surface is free of vane passage and unsegmented.

27. The transcatheter blood pump according to claim 23, wherein the first bearing surface comprises a first curved portion and a first flat portion, and the second bearing surface comprises a second curved portion and a second flat portion.

28. The transcatheter blood pump according to claim 27, wherein the first flat portion extends radially outward from the first curved portion, and the second flat portion extends radially outward from the second curved portion.

29. The transcatheter blood pump according to claim 23, wherein the impeller internal passage comprises a helical path that winds about the axis of rotation of the impeller, the helical path configured to enhance blood flow therethrough.

30. The transcatheter blood pump according to claim 23, further comprising a first magnetic element associated with the impeller, and a second magnetic element associated with the electric motor, the first magnetic element configured to magnetically couple with the second magnetic element to transmit torque between the impeller and the electric motor, wherein the first magnetic element is disposed at least partially within at least one vane of the plurality of vanes.
